(19) United States
(12) Patent Application Publication
(10) Pub. No.: US 2010/0166857 A1
(43) Pub. Date: Jul. 1, 2010

(54) PHARMACEUTICAL DOSAGE FORMS AND METHODS OF MANUFACTURING SAME

(76) Inventors: Dong Yan, Palo Alto, CA (US); Lee Foong Siew, Dublin, CA (US); Richard Ting, Danville, CA (US); Tzu-Yuan Chen, San Jose, CA (US); Mahendra Sudhakar Pawar, Santa Rosa, CA (US)

Correspondence Address: GREENBERG TRAURIG, LLP, MET LIFE BUILDING, 200 PARK AVENUE, NEW YORK, NY, 10166, US

(21) Appl. No.: 12/649,943

(22) Filed: Dec. 30, 2009

Related U.S. Application Data
(60) Provisional application No. 61/141,472, filed on Dec. 30, 2008.

(51) Int. Cl. A61K 9/26; A61K 9/50; A61K 31/216; A61P 9/10; A61P 3/06; B05D 1/02

(52) U.S. Cl. 424/465; 424/494; 424/497; 514/543; 427/2.15

(57) ABSTRACT

The invention provides solid dispersions of at least one insoluble active pharmaceutical ingredient, pharmaceutical dosage forms comprising the solid dispersions, and methods of manufacturing same. In an embodiment, a solid dispersion of the present invention includes a plurality of coated particles comprising inert particles with a coating, wherein the coating comprises an insoluble active pharmaceutical ingredient dispersed in a hydrophilic polymer, and wherein the inert particles comprise nonpareils; and a plurality of granules comprising an insoluble active pharmaceutical ingredient with at least one pharmaceutically acceptable excipient. In an embodiment, the insoluble active pharmaceutical ingredient in the coating and the insoluble active pharmaceutical ingredient of the granules are the same type. A solid dispersion of the present invention may optionally be encapsulated in capsules or compressed into a tablet. Also disclosed are methods of making solid dispersions and methods of reducing plasma triglyceride and increasing high-density lipoprotein employing the solid dispersion.

### RELATED APPLICATIONS

This application claims the benefit of and priority to U.S. Provisional Application Ser. No. 61/141,472, filed Dec. 30, 2008, the entirety of this application is hereby incorporated herein by reference.

### BACKGROUND

Product development scientists often encounter difficulties in solving the problem of poor water solubility of drug candidates in the development of pharmaceutical dosage forms. More than one-third of the drugs listed in the United States Pharmacopeia (USP) fall into the poorly water-soluble or water-insoluble categories. Active ingredients with poor water solubility provide formulation obstacles, as they tend to be eliminated from the gastrointestinal tract prior to absorption, leading to poor bioavailability. The availability to the biologic system of a pharmaceutical product is integral to the goals of dosage form design and paramount to the effectiveness of the medication. The study of a drug's bioavailability depends on the drug's absorption or entry into the systemic circulation. The rate and extent to which a drug in a dosage form becomes available for biologic absorption or use depends on the materials in the formulation and on the method of manufacture. Thus, the same drug when formulated in different dosage forms may be found to possess different bioavailability characteristics and hence exhibit different clinical effectiveness. Furthermore, two seemingly identical or equivalent products of the same drug in the same dosage strength and in the same dosage form but differing in formulative materials or method of manufacture may vary widely in bioavailability and thus in clinical effectiveness.

### SUMMARY

Pharmaceutical dosage forms and methods of manufacturing same are disclosed herein.

In an embodiment, the present invention relates to a solid dispersion that includes a plurality of coated particles comprising inert particles with a coating, wherein the coating comprises at least one insoluble active pharmaceutical ingredient dispersed in a hydrophilic polymer, and wherein the inert particles comprise nonpareils; and a plurality of granules comprising at least one insoluble active pharmaceutical ingredient with at least one pharmaceutically acceptable excipient. In an embodiment, the hydrophilic polymer of the coating is hypromellose. In an embodiment, the insoluble active pharmaceutical ingredient in the coating and the insoluble active pharmaceutical ingredient of the granules are the same type. In an embodiment, the insoluble active pharmaceutical ingredient in the coating and the insoluble active pharmaceutical ingredient of the granules are in micronized form. In an embodiment, the coating further includes a surfactant. In an embodiment, the coated particles are further coated with a seal coating. In an embodiment, the solid dispersion is formed into a pharmaceutical dosage form. In an embodiment, the pharmaceutical dosage form is suitable for oral administration. In an embodiment, the plurality of granules are present in the pharmaceutical dosage form at a percentage ranging from about 0% to about 65%. In an embodiment, the plurality of granules are present in the pharmaceutical dosage form at a percentage ranging from about 35% to about 65%.

In an embodiment, the present invention relates to a solid dispersion that includes a plurality of coated particles comprising inert particles with a coating, wherein the coating comprises fenofibrate dispersed in a hydrophilic polymer, and wherein the inert particles comprise nonpareils; and a plurality of granules comprising micronized fenofibrate with at least one pharmaceutically acceptable excipient. In an embodiment, the hydrophilic polymer of the coating is hypromellose. In an embodiment, the granules are present at a percentage ranging from about 40% to about 60%, by weight, based on the total combined weight of the solid dispersion. In an embodiment, the fenofibrate of the coating is further dispersed in a surfactant. In an embodiment, the surfactant is sodium lauryl sulfate. In an embodiment, the coated particles are further coated with a seal coating. In an embodiment, the plurality of granules are present in the pharmaceutical dosage form at a percentage ranging from about 0% to about 65%. In an embodiment, the plurality of granules are present in the solid dispersion at a percentage ranging from about 35% to about 65%. In an embodiment, the solid dispersion is formed into a pharmaceutical dosage form. In an embodiment, the pharmaceutical dosage form is in a unit dose comprising from about 10 mg to about 200 mg fenofibrate as an immediate release tablet. In an embodiment, the pharmaceutical dosage form comprises a sufficient amount of granules in order to decrease relative bioavailability of fenofibrate as compared to a pharmaceutical dosage form that does not include granules.

In an embodiment, the present invention discloses a pharmaceutical dosage form that includes a plurality of active seeds blended with pharmaceutical excipients, wherein the active seeds comprise inert particles coated with a dispersion of fenofibrate, hypromellose and a stabilizer prepared by using a fluidized bed coating process, wherein the pharmaceutical dosage form is sufficiently designed such that about 65% of the fenofibrate is dissolved within 10 minutes when tested with a USP paddle method at 50 rpm, 1000 mL of Simulated Gastric Fluid (SGF) in 1% Tween 80 pH 1.2 at about 37° C. In an embodiment, a ratio of the hypromellose to fenofibrate is from about 10:1 to about 1:10. In an embodiment, the coating on the active seeds further comprises a surfactant. In an embodiment, the surfactant is sodium lauryl sulfate. In an embodiment, the coated inert particles are further coated with a seal coating. In an embodiment, the pharmaceutical dosage form is in a unit dose comprising from about 10 mg to about 200 mg fenofibrate. In an embodiment, the pharmaceutical dosage form is in a unit dose comprising from about 10 mg to about 200 mg fenofibrate as a single matrix tablet. In an embodiment, the pharmaceutical dosage form is in a unit dose comprising from about 10 mg to about 200 mg fenofibrate as an immediate release tablet. In an embodiment, the pharmaceutical dosage form provides a mean Cₘₐₓ of fenofibric acid of about 9.0 μg/mL to about 12.0 μg/mL after administration of a single dose to a patient population in the fasted state based on a 145 mg dose of fenofibrate. In an embodiment, the pharmaceutical dosage form provides a mean Cₘₐₓ of fenofibric acid of about 8.0 μg/mL to about 10.0 μg/mL after administration of a single dose to a patient population in the fed state based on a 145 mg dose of fenofibrate. In an embodiment, the pharmaceutical dosage form provides a Tₘₐₓ of from about 1 hour to about 3 hours after single dose administration to healthy volunteers under fasted state. In an embodiment, the pharmaceutical dosage form provides a Tₘₐₓ of from about 3 hours to about 5 hours after single dose administration to healthy volunteers under fed state.

In an embodiment, a solid dispersion of the present invention comprises fenofibrate. In an embodiment, at least about 50%, at least about 60%, or at least about 70% of the fenofibrate is dissolved within 20 minutes when tested with a USP paddle method at 50 rpm, 1000 mL of Simulated Gastric Fluid (SGF) in 1% Tween 80 pH 1.2 at about 37° C.

In an embodiment, a solid dispersion of the present invention comprises fenofibrate. In an embodiment, at least about 60%, at least about 70%, or at least about 80% of the fenofibrate is dissolved within 40 minutes when tested with a USP paddle method at 50 rpm, 1000 mL of Simulated Gastric Fluid (SGF) in 1% Tween 80 pH 1.2 at about 37° C.

In an embodiment, a solid dispersion of the present invention comprises fenofibrate. In an embodiment, at least about 85%, at least about 90%, or at least about 95% of the fenofibrate is dissolved within 10 minutes when tested with a USP paddle method at 75 rpm, 900 mL of medium with 0.75% of sodium lauryl sulfate in water at about 37° C.

In an embodiment, a solid dispersion of the present invention comprises fenofibrate. In an embodiment, at least about 90%, at least about 95%, or at least about 100% of the fenofibrate is dissolved within 20 minutes when tested with a USP paddle method at 75 rpm, 900 mL of medium with 0.75% of sodium lauryl sulfate in water at about 37° C.

In an embodiment, a pharmaceutical dosage form of the present invention provides increased bioavailability of an insoluble active pharmaceutical ingredient as compared to another formulation of the same active pharmaceutical ingredient. In an embodiment, a pharmaceutical dosage form of the present invention can be administered to a patient at a therapeutically effective amount to treat a condition selected from the group consisting of hypercholesterolemia, hypertriglyceridemia, coronary heart disease, cardiovascular disease, peripheral vascular disease, symptomatic carotid artery disease, mixed dyslipidemia, and increases risk of pancreatitis.

In an embodiment, a pharmaceutical dosage form of the present invention in a unit dose comprising about 145 mg fenofibrate in the form of a single matrix tablet is administered to a patient in the fasted state and provides a mean Cₘₐₓ of fenofibric acid of about 4 μg/mL to about 14 μg/mL, about 6 μg/mL to about 12 μg/mL, or about 8 μg/mL to about 10 μg/mL.

In an embodiment, a pharmaceutical dosage form of the present invention in a unit dose comprising about 145 mg fenofibrate in the form of a single matrix tablet is administered to a patient in the fed state and provides a mean Cₘₐₓ of fenofibric acid of about 5 μg/mL to about 12 μg/mL, about 6 μg/mL to about 11 μg/mL, or about 7 μg/mL to about 9 μg/mL.

In an embodiment, a pharmaceutical dosage form of the present invention in a unit dose comprising about 145 mg fenofibrate in the form of a single matrix tablet is administered to a patient in the fed or fasted state and provides a mean Tₘₐₓ of fenofibric acid from about 4 to about 10 hours, from about 5 to about 9 hours, or from about 6 to about 8 hours.

In an embodiment, a pharmaceutical dosage form of the present invention in a unit dose comprising about 120 mg fenofibrate in the form of a single matrix tablet is administered to a patient in the fasted state and provides a mean Cₘₐₓ of fenofibric acid of about 3.0 μg/mL to about 9.0 μg/mL, about 4.0 μg/mL to about 8.0 μg/mL, or about 5.0 μg/mL to about 7.0 μg/mL.

In an embodiment, a pharmaceutical dosage form of the present invention in a unit dose comprising about 120 mg fenofibrate in the form of a single matrix tablet is administered to a patient in the fed state and provides a mean Cₘₐₓ of fenofibric acid of about 4.0 μg/mL to about 10.0 μg/mL, about 5.0 μg/mL to about 9.0 μg/mL, or about 6.0 μg/mL to about 8.0 μg/mL.

In an embodiment, a pharmaceutical dosage form of the present invention in a unit dose comprising about 120 mg fenofibrate in the form of a single matrix tablet is administered to a patient in the fed or fasted state and provides a mean Tₘₐₓ of fenofibric acid from about 0.5 to about 5.0 hours, from about 1.0 to about 4.5 hours, or from about 2.0 to about 3.5 hours.

In an embodiment, the present invention discloses a method of treating hypercholesterolemia in a patient comprising administering a therapeutically effective amount of a pharmaceutical dosage form of the present invention. In an embodiment, after administration of the pharmaceutical dosage form, increased bioavailability of an insoluble active pharmaceutical ingredient is observed as compared to another formulation of the same active pharmaceutical ingredient.

In an embodiment, the present invention discloses a method of treating hypertriglyceridemia in a patient comprising administering a therapeutically effective amount of a pharmaceutical dosage form of the present invention. In an embodiment, after administration of the pharmaceutical dosage form, increased bioavailability of an insoluble active pharmaceutical ingredient is observed as compared to another formulation of the same active pharmaceutical ingredient.

In an embodiment, the present invention discloses a method of preparing a solid dispersion that includes dissolving an insoluble active pharmaceutical ingredient and a hydrophilic polymer in a suitable solvent to form a solution; preparing a plurality of active seeds by spray coating a plurality of inert particles with the solution using a fluid bed coating process; preparing a plurality of active granules by wet granulating a mixture of an insoluble active pharmaceutical ingredient with at least one pharmaceutical acceptable excipient; and blending the plurality of active seeds and the plurality of active granules. In an embodiment, the insoluble active pharmaceutical ingredient coating the inert particles and the insoluble pharmaceutical ingredient of the granules are of a same type. In an embodiment, the insoluble active pharmaceutical ingredient coating the inert particles and the insoluble active pharmaceutical ingredient of the granules are in a micronized form. In an embodiment, the insoluble active pharmaceutical ingredients are fenofibrate. In an embodiment, the insoluble active pharmaceutical ingredient is further dispersed in a surfactant. In an embodiment, the coated inert particles are further coated with a seal coating. In an embodiment, the plurality of active granules are present in the solid dispersion at a percentage ranging from about 35% to about 65%.

In an embodiment, the present invention relates to methods of improving the solubility of an active pharmaceutical ingredient, for example, fenofibrate.

### BRIEF DESCRIPTION OF THE DRAWINGS

FIG. 1 is a flow chart showing an embodiment of a method of preparing a solid dispersion of the present invention and a pharmaceutical dosage form comprising the solid dispersion.

FIG. 2 is a flow chart showing an embodiment of a method of preparing a fenofibrate solid dispersion of the present invention and a pharmaceutical dosage form comprising the solid dispersion.

FIGS. 3A-3F show polarized micrographs depicting the morphology of drug/polymer films. FIG. 3A shows a polarized micrograph depicting the morphology of drug/Pharmacoat® 603 films. FIG. 3B shows a polarized micrograph depicting the morphology of drug/Kollidon® VA 64 PVP films. FIG. 3C shows a polarized micrograph depicting the morphology of drug/PEG 6000 films. FIG. 3D shows a polarized micrograph depicting the morphology of drug/Klucel® HPC Grade LF films. FIG. 3E shows a polarized micrograph depicting the morphology of drug/Poloxamer 188 films. FIG. 3F shows a polarized micrograph depicting the morphology of drug/HPMCAS films.

FIG. 4 shows a graph of the in-vitro dissolution profiles of various coatings of the present invention for use in preparing a solid dispersion of the present invention.

FIG. 5 shows a graph of the in-vitro dissolution profiles of various dosage forms equivalent to 145 mg fenofibrate.

FIG. 6 shows the linear mean average plasma concentration of fenofibric acid versus time for 12 patients dosed with pharmaceutical dosage forms of the present invention equivalent to 145 mg fenofibrate and Tricor® 145 mg tablets under fasted conditions.

FIG. 7 shows the linear mean average plasma concentration of fenofibric acid versus time for 12 patients dosed with pharmaceutical dosage forms of the present invention equivalent to 145 mg fenofibrate and Tricor® 145 mg tablets under fed conditions.

FIG. 8 shows a graph of the in-vitro dissolution profiles of various pharmaceutical dosage forms of the present invention.

FIG. 9 shows the linear mean average plasma concentration of fenofibric acid versus time for 12 patients dosed with pharmaceutical dosage forms of the present invention equivalent to 120 mg fenofibrate and Fenoglide® 120 mg tablets under fasted conditions.

FIG. 10 shows the linear mean average plasma concentration of fenofibric acid versus time for 12 patients dosed with pharmaceutical dosage forms of the present invention equivalent to 120 mg fenofibrate and Fenoglide® 120 mg tablets under fed conditions.

FIG. 11 shows the linear mean average plasma concentration of fenofibric acid versus time for 12 patients dosed with pharmaceutical dosage forms of the present invention equivalent to 120 mg fenofibrate and Fenoglide® 120 mg tablets under fasted conditions.

FIG. 12 shows the linear mean average plasma concentration of fenofibric acid versus time for 12 patients dosed with pharmaceutical dosage forms of the present invention equivalent to 120 mg fenofibrate and Fenoglide® 120 mg tablets under fed conditions.

### DETAILED DESCRIPTION

According to aspects illustrated herein, the present invention is directed to solid dispersions of at least one insoluble active pharmaceutical ingredient (API), pharmaceutical dosage forms comprising the solid dispersions, and methods of manufacturing same. In an embodiment, a pharmaceutical dosage form of the present invention comprises a plurality of drug-coated seeds and a plurality of drug granules in a single tablet. In an embodiment, the single tablet is an immediate release tablet.

As used herein, “about” will be understood by persons of ordinary skill in the art and will vary to some extent based upon the context in which it is used. If there are uses of the term which are not clear to persons of ordinary skill in the art given the context in which it is used, “about” will mean up to plus or minus 10% of the particular term.

As used herein, the terms “active ingredient”, “AI”, “active pharmaceutical ingredient” and “API” refer to the substance in a pharmaceutical drug that is biologically active. An API suitable for use with the present invention can be selected from a variety of known classes of drugs or diagnostic ingredients. In an embodiment, the API is one which is insoluble, including, but not limited to, analgesics, anti-inflammatory agents, anthelmintics, anti-arrhythmic ingredients, antibiotics (including penicillins), anticoagulants, antihypercholesterolemia ingredients (including fibrates and fenofibric acid), antidepressants, antidiabetic ingredients, antiepileptics, antihistamines, antihypertensive ingredients, antimuscarinic ingredients, antimycobacterial ingredients, antineoplastic ingredients, immunosuppressants, antithyroid ingredients, antiviral ingredients, anxiolytic sedatives (hypnotics and neuroleptics), astringents, beta-adrenoceptor blocking ingredients, blood products and substitutes, cardiac inotropic ingredients, contrast media, corticosteroids, cough suppressants (expectorants and mucolytics), diagnostic ingredients, diagnostic imaging ingredients, diuretics, dopaminergics (antiparkinsonian ingredients), haemostatics, immunological ingredients, lipid regulating ingredients, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid ingredients, vasodilators, and xanthines. Specific examples of insoluble APIs that can be utilized in the present invention include, but are not limited to, cox-2 inhibitors, anti-inflammatory drugs such as nimesulide, piroxicam, naproxene, ketoprofen, ibuprofen and diacerheine, antifungal drugs such as griseofulvin, itraconazole, fluconazole, miconazole and ketonazole, bronchodilators/anti-asthmatic drugs such as zafrilukast, salbutamol, beclomethasone, flunisolide, clenbuterol, salmeterol and budesonide, steroids such as estradiol, estriol, progesterone, megestrol acetate, medroxyprogesterone acetate, antihypertensive/antithrombotic/vasodilator drugs such as nefedipine, nicergoline, nicardipine, lisinopril, enalapril, nicorandil, celiprolol and verapamil, benzodiazepines such as temazepam, diazepam, lorazepam, fluidiazepam, medazepam and oxazolam, anti-migraine drugs such as zolmitriptan and sumatriptan, antilipoproteinemic drugs such as lovastatin, atorvastatin, fluvastatin, and simvastatin, anti-viral/antibacterial drugs such as tosufloxacin, ciprofloxacin, ritonavir, saquinavir, nelfinavir, acyclovir and indinavir, immunodepressant drugs such as lacrolimus, rapamycine and didanisine, anti-histaminic drugs such as loratadine, antitumour drugs such as etoposide, bicalutamide, tamoxifen, doclitaxel and paclitaxel, anti-psychotic drugs such as risperidone, antiosteoporotic drugs such as raloxifene, anti-convulsant drugs such as carbamazepin and phenyloin, analgetic/narcotic drugs such as oxycodone, hydrocodone, morphine and butorpanol, muscle relaxants such as tinazadine, and anti-ulcerative drugs such as famotidine.

When APIs described in the invention contain relatively basic functionalities, acid addition salts can be obtained by contacting the neutral form of such compounds with a sufficient amount of the desired acid, either neat (without solvent) or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, ptolylsulfonic, citric, tartaric, methanesulfonic, and the like. Also included are salts of amino acids such as arginate and the like, and salts of organic acids like glucuronic or galactunoric acids and the like. Certain specific therapeutic ingredients contain both basic and acidic functionalities that allow the compounds to be converted into either base or acid addition salts.

As used herein, the term “active seed” refers to an inert particle that has been coated with a solution, wherein the solution comprises at least one insoluble active pharmaceutical ingredient and at least one hydrophilic polymer dissolved in a suitable solvent. In an embodiment, the solution is coated onto the inert particle using a spray coating process. In an embodiment, the spray coating process is a fluid bed coating process having a Wurster insert.

As used herein, the term “bioavailability” is used to describe the fraction of an administered dose of unchanged drug that reaches the systemic circulation, one of the principal pharmacokinetic properties of drugs. By definition, when a medication is administered intravenously, its bioavailability is 100%. However, when a medication is administered via other routes (such as orally), its bioavailability decreases (due to incomplete absorption and first-pass metabolism). The term “relative bioavailability” measures the bioavailability (estimated as area under the curve, or AUC) of a certain drug when compared with another formulation of the same drug, usually an established standard, or through administration via a different route. For FDA approval, a generic manufacturer must show that the 90% confidence interval for the ratio of the mean response (usually AUC and Cₘₐₓ) of its product to that of the “Brand Name drug” is within the limits of 0.8 to 1.25 at the 0.05 level of significance.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “C.” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} is the calculated area under the curve of plasma concentration versus time from time 0 to time infinity.

As used herein, the term “bioequivalence” refers to the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. In determining bioequivalence, for example, between two products such as a commercially-available branded product and a potential to-be-marketed generic product, pharmacokinetic studies are conducted whereby each of the preparations are administered in a cross-over study to volunteer subjects, generally healthy individuals but occasionally in patients. Serum/plasma samples are obtained at regular intervals and assayed for parent drug (or occasionally metabolite) concentration. For a pharmacokinetic comparison, the plasma concentration data are used to assess key pharmacokinetic parameters such as area under the curve (AUC), peak concentration (Cₘₐₓ), time to peak concentration (Tₘₐₓ), and absorption lag time (t_{lag}). Under U.S. FDA guidelines, two products (e.g., an inventive composition and TriCor® 145 mg Manufactured for Abbott Laboratories, North Chicago, Ill. 60064, U.S.A. or Fenoglide® 40 mg or 120 mg Manufactured for Sciele Pharma, Inc., Atlanta, Ga. 30328, U.S.A.) or methods (e.g., dosing under non-fasted versus fasted conditions) are bioequivalent if the 90% Confidence Intervals (CI) for the ratios of a log transformed geometric mean of AUC_{0-∞} for the first product or method compared to the second product or method, and Cₘₐₓ for the first product or method compared to the second product or method, are within 0.80 to 1.25 (Tₘₐₓ measurements are not relevant to bioequivalence for regulatory purposes). To show bioequivalency between two compositions or methods pursuant to Europe's EMEA guidelines, the 90% CI for the ratios of a log transformed geometric mean of AUC_{0-∞} for the first product or method compared to the second, must be within 0.80 to 1.25 and the 90% CI for the ratios of a log transformed geometric mean of Cₘₐₓ for the first product or method compared to the second must be within 0.70 to 1.43.

The term “effective amount” means an amount of an API in a pharmaceutical dosage form according to the present invention effective in producing the desired therapeutic effect.

Food is typically a solid food with sufficient bulk and fat content that it is not rapidly dissolved and absorbed in the stomach. In one embodiment, “food” is a meal, such as breakfast, lunch, or dinner. The terms “taken with food”, “fed” and “non-fasted” are equivalent and are as given by FDA guidelines and criteria. In one embodiment, “with food” means that the dosage form is administered to a patient between about 30 minutes prior to about 2 hours after eating a meal. In another embodiment, “with food” means that the dosage is administered at substantially the same time as eating the meal.

The terms “without food,” “fasted,” and “an empty stomach” are equivalent and are as given by FDA guidelines and criteria. In one embodiment, “fasted” means the condition of not having consumed solid food for at least about 1 hour prior or at least about 2 hours after such consumption. In another embodiment, “fasted” means the condition of not having consumed solid food for at least about 1 hour prior to at least about 2 hours after such consumption.

For the purposes of biostudy and the determination of bioequivalence, a “fasted patient” means a patient who does not eat any food, i.e., fasts, for at least 10 hours before the administration of a dosage form of active agent and who does not eat any food and continues to fast for at least 4 hours after the administration of the dosage form. The dosage form is administered with water during the fasting period, and water can be allowed ad libitum after 2 hours.

For the purposes of biostudy and the determination of bioequivalence, a “non-fasted patient” means a patient who fasts for at least 10 hours overnight and then consumes an entire test meal within 30 minutes of first ingestion. The dosage form is administered with water at 30 minutes after first ingestion of the meal. No food is then allowed for at least 4 hours post-dose. Water can be allowed ad libitum after 2 hours.

“Fenofibrate” as employed herein refers to fenofibrate, its derivatives, prodrugs, active metabolites, and/or its polymorphs, solvates, hydrates, enantiomers, racemates and mixtures thereof. Further, it also includes amorphous or crystalline polymorphic forms of fenofibrate, and mixtures thereof. The chemical name for fenofibrate is 2-[4-(4-chlorobenzoyl)phenoxy]-2-methyl-propanoic acid, 1-methylethyl ester. The empirical formula is C₂₀H₂₁O₄Cl. Fenofibrate is currently used in the treatment of endogenous hyperlipidaemias, hypercholesterolaemias, and hypertriglyceridaemias in adults. Fenofibric acid, the active metabolite of fenofibrate, has been shown to produce reductions in total cholesterol, LDL cholesterol, apolipoprotein B, total triglycerides and triglyceride rich lipoprotein (VLDL) in treated patients. Also, treatment with fenofibrate has been shown to produce an increase in high-density lipoprotein (HDL) and apoproteins apoAI and apoAII.

As used herein, the term “fibrate” means any of the fibric acid derivatives useful in the methods described herein, including, but not limited to, bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate and theofibrate. Generally, fibrates are used to treat conditions such as hypercholesterolemia, mixed lipidemia, hypertriglyceridemia, coronary heart disease, and peripheral vascular disease (including symptomatic carotid artery disease), and prevention of pancreatitis. A particular fibrate, fenofibrate, may help prevent the development of pancreatitis (inflammation of the pancreas) caused by high levels of triglycerides in the blood. Fibrates are known to be useful in treating renal failure. Fibrates may also be used for other indications where lipid regulating agents are typically used.

As used herein, the term “fluid bed coating” refers to a uniform, continuous product coating process in which the coating step and the drying step take place in one machine. With fluid bed coating, particles are fluidized and the coating fluid sprayed on and dried. Small droplets and a low viscosity of the spray medium ensure an even product coating. In an embodiment, the fluid bed coating employed in the fabrication of a solid dispersion of the present invention is a bottom spray coating process (Wurster coating). In the Wurster process, a complete sealing of the surface can be achieved with a low usage of coating substance. The spray nozzle is fitted in the base plate resulting in a spray pattern that is concurrent with the air feed. By using a Wurster cylinder and a base plate with different perforations, the particles to be coated are accelerated inside the Wurster tube and fed through the spray cone concurrently. As the particles continue travelling upwards, they dry and fall outside the Wurster tube back towards the base plate. They are guided from the outside back to the inside of the tube where they are once again accelerated by the spray. This produces an extremely even film. Particles of different sizes are evenly coated.

“Inert Particles” used in the present invention include nonpareils in the form of sugar beads, starch beads or sugar/starch beads manufactured by any process known in the art of making pellets, e.g., extrusion spheronization, centrifugal coating, muster coating, etc. The inert particles may also be a cellulosic material such as microcrystalline cellulose or a non-toxic plastic material. The size of the inert particles can be from about 10 mesh to about 100 mesh, from about 25 mesh to about 40 mesh, or about 60 mesh to about 80 mesh.

“Immediate release” refers to a pharmaceutical dosage form which releases the API substantially immediately upon contact with gastric juices and will result in substantially complete dissolution within about 1 hour. When used in association with the dissolution profiles discussed herein, the term “immediate release” refers to that portion of a pharmaceutical dosage form made according to the present invention which delivers the API over a period of time less than 1 hour.

As used herein, the phrase “insoluble” includes “sparingly soluble”, “slightly soluble”, “very slightly soluble”, or “practically insoluble” in water. According to the United States Pharmacopeia (USP) XXI (page 1441) an active ingredient is described as “sparingly soluble” if one part of solute (active ingredient) requires from 30-100 parts of solvent to dissolve it; “slightly soluble” if one part of solute requires from 100-1000 parts of solvent to dissolve it; “very slightly soluble” if one part of solute requires from 1,000-10,000 parts of solvent to dissolve it; and “practically insoluble” if one part of solute requires more than 10,000 parts of solvent to dissolve it.

As used herein, the term “patient” means any mammal including humans.

In some embodiments, a “solid dispersion” refers to a plurality of solid products, wherein the solid products comprise a hydrophilic matrix and an insoluble API coated on an inert particle using a fluidized-bed system, wherein the solid products may optionally include a seal coating. In an embodiment, fenofibrate, hypromellose (HPMC), and sodium lauryl sulfate are dissolved in a mixture of acetone and water (4:1) and spray coated on sugar spheres using a fluidized-bed coating system to create a plurality of solid products (a solid dispersion), followed by seal coating of the solid products with a mixture of acetone and water (4:1) having dispersed therein amino methacrylate copolymer, talc, and magnesium stearate. In some embodiments, a solid dispersion of the present invention refers to a plurality of solid products as described above, and further includes a plurality of drug granules comprising an insoluble API and one or more pharmaceutically acceptable excipients. In an embodiment, a solid dispersion of the present invention includes a plurality of solid products and a plurality of drug granules, wherein the drug granules are present at a percentage ranging from about 0% to about 60% based on the weight of API. For example, a solid dispersion of the present invention having “60% micronized API” refers to a solid dispersion wherein 60% by weight of the API is from the granules and 40% by weight of the API is from the solid products. A pharmaceutical dosage form of the present invention may comprise a solid dispersion disclosed herein. Pharmaceutical dosage forms of the present invention include, but are not limited to, those suitable for oral, rectal, topical and buccal (e.g. sublingual) administration.

In an embodiment, a solid dispersion of the present invention includes at least one insoluble API that is an antihypercholesterolemia ingredient. In an embodiment, the antihypercholesterolemia ingredient is a fibrate, such as fenofibrate or fenofibric acid. In embodiments where fenofibrate is the API, fenofibrate may be present in an amount of from about 10 mg to about 500 mg, from about 25 mg to about 400 mg, from about 50 mg to about 350 mg, from about 75 mg to about 300 mg, or from about 100 mg to about 250 mg. In certain embodiments, the fenofibrate is present in an amount selected from the group consisting of 40 mg, 43 mg, 48 mg, 50 mg, 54 mg, 67 mg, 100 mg, 107 mg, 120 mg, 130 mg, 134 mg, 145 mg, 150 mg, 160 mg and 200 mg.

As used herein, the term “therapeutically effective amount” refers to the amount/dose of an active pharmaceutical ingredient or a pharmaceutical dosage form that is sufficient to produce an effective response (i.e., a biological or medical response of a tissue, system, animal or human sought by a researcher, veterinarian, medical doctor or other clinician) upon administration to a patient. The “therapeutically effective amount” will vary depending on inter alia the disease and its severity, and the age, weight, physical condition and responsiveness of the patient to be treated.

The USP paddle method refers to the Paddle and Basket Method as described in United States Pharmacopoeia, Edition XXII (1990).

The development of solid dispersions as a practically viable method to enhance bioavailability of poorly water-soluble drugs overcame the limitations of previous approaches such as salt formation, solubilization by cosolvents, and particle size reduction. Conventional methods for preparing solid dispersions include solvent-based, fusion-melt and hybrid fusion-solvent methods. The solvent-based method commonly uses a co-solvent to intimately disperse or dissolve the active ingredient(s) and carrier molecules together, and then evaporates the solvent by evaporation. Then, the solid dispersion is collected as a powdered mass. The fusion-melt involves melting the ingredient(s) and the carrier components together at temperatures at or above the melting point of all components. In the fusion process, the ingredient and the carrier are blended in a suitable mixer. The process includes heating and melting the blend, followed by cooling the molten mixture rapidly to provide a congealed mass. This mass is then milled to produce powders at desired particle size ranges. Sometimes, a hybrid fusion-solvent method is used if thermal instability and immiscibility between the ingredient(s) and the carrier are present. In the hybrid fusion-solvent method, the ingredient(s) are dissolved in a small quantity of organic solvent and added to the molten carrier. The solvent is then evaporated to generate the mass. The mass is milled to produce powder at desired particle size ranges. The limitations of these technologies have been a drawback for the commercialization of solid dispersions. The limitations include, but are not limited to, laborious and expensive methods of preparation, reproducibility of physicochemical characteristics, difficulty in incorporating into formulation of dosage forms, scale-up of manufacturing process, and stability of the drug and vehicle.

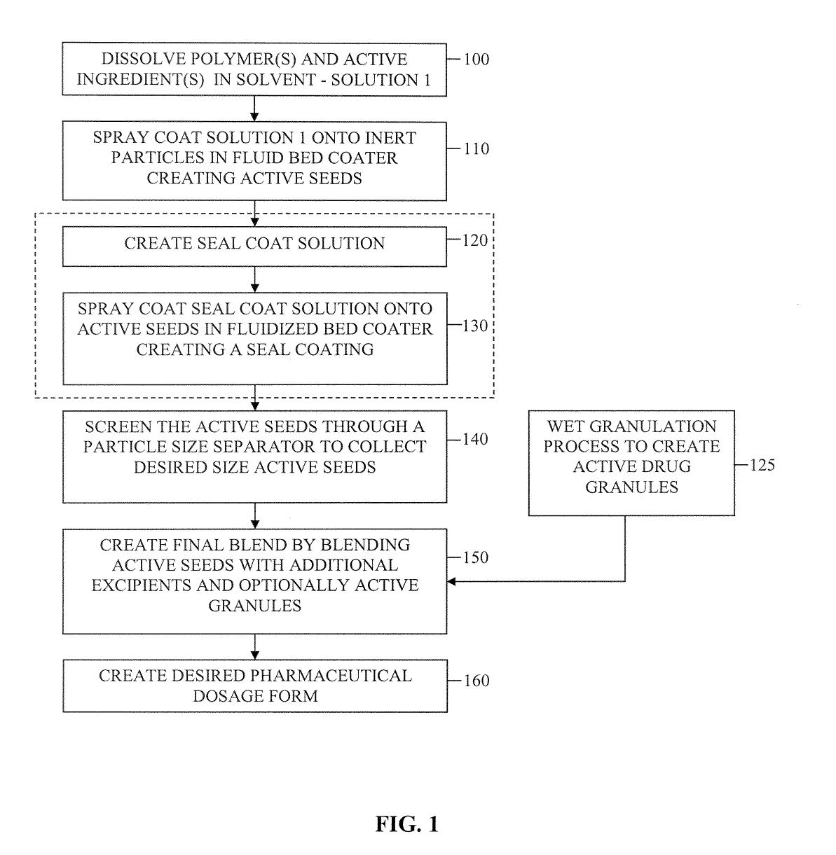

An embodiment of a method of preparing a solid dispersion of the present invention and a pharmaceutical dosage form comprising the solid dispersion is illustrated in FIG. 1. As illustrated in FIG. 1, at least one polymer and at least one API are dissolved in a solvent to form solution 1, step 100. Solubilizing agent(s) (for example, a surfactant) may be added to solution 1 as necessary to increase the solubility of the API in the solvent. The dissolution profile of the API in solution 1 can be adjusted by varying the amount and type of polymer(s) and surfactant(s) included. For example, there is a direct relationship between the amount of polymer and dissolution of the API. Therefore, as the ratio of polymer to API increases, the apparent solubility and bioavailability also increases. In certain embodiments, the ratio of polymer to API is from about 20:1 to about 1:20; from about 15:1 to about 1:15; from about 10:1 to about 1:10; from about 5:1 to about 1:5; from about 2:1 to about 1:2; from about 1.5:1 to about 1:1.5. In an embodiment, the ratio of polymer to API is 1.5:1.

Using a fluid bed coating process, solution 1 is coated onto inert particles and dried, creating active seeds, step 110. In an embodiment, the coating is a drug-loaded polymer film. Optionally, a seal coating may be applied to the active seeds to improve the stability of the active seeds (steps 120 and 130). The active seeds, with or without the seal coating, are then screened through a particle size separator to collect the desired size active seeds, step 140. In an embodiment, active seeds between 40 mesh (about 420 μm, 0.42 mm) and 60 mesh (about 250 μm, 0.25 mm) are collected. In an embodiment, the size of the active seeds are about 0.30 mm. The method may optionally include step 125, where micronized API undergoes a wet-granulation process (WG) to create active granules. In step 150, a final blend is created by blending the active seeds with pharmaceutical excipients and optionally the active granules. A desired pharmaceutical dosage form can be created, step 160. In an embodiment, step 125 is performed to adjust the bioavailability of the final blend, and thus the pharmaceutical dosage form. In an embodiment, the addition of step 125 to the method results in the reduction in the bioavailability of the final blend, and thus the bioavailability of the pharmaceutical dosage form. In an embodiment, the pharmaceutical dosage form comprises a plurality of drug-coated seeds and a plurality of drug granules in a single matrix tablet. In an embodiment, the pharmaceutical dosage form is an immediate release tablet. Controlled-release formulations which control the rate of release or the time of release, or both, of the active ingredient are also contemplated and include sustained-, pulsed-, dual- and delayed-release formulations. Controlled-release formulations may be prepared by any method known by those of skill in the art, and include, e.g., layered tablets, controlled-release matrices, rate-controlling membrane coatings, impermeable membranes with apertures, semi-permeable membranes, etc.

In an embodiment, surfactants are used to increase the solubility of the API in the solvent and to stabilize solution 1 to prevent recrystalliztion of the API through steric or ionic interactions. The surfactants utilized in the present invention can be selected from the group consisting of anionic surfactants, cationic surfactants, amphoteric surfactants, non-ionic surfactants and mixture thereofs. Examples of surfactants include, but are not limited to, sodium lauryl sulfate (SLS), sodium docusate, Vitamin E TPGS, Labrasol, Cremophor, Solutol HS-15, Tween, and Pluronic. The amount of surfactant in solution 1 may have an affect on the preparation of the solid dispersion. For example, if an increased amount of surfactant is used, surfactant molecules may self-assemble to develop a separated phase. Further, if a decreased amount of surfactant is used, crystal growth in solid state may not be effectively inhibited. Therefore, an optimal concentration should be present. The surfactant concentration can depend on the amount and the hydrophilicity of the polymer, and the amount and hydrophobicity of the API. In an embodiment, the ratio of API to surfactant is from about 0.5:20 to about 20:0.5; from about 1:10 to about 10:1. In an embodiment, the ratio of polymer to surfactant is from about 0.5:50 to about 50:0.5; from about 4:10 to about 10:4.

Examples of anionic surfactants include, but are not limited to, monovalent alkyl carboxylates, acyl lactylates, alkyl ether carboxylates, N-acyl sarcosinates, polyvalent alkyl carbonates, N-acyl glutamates, tatty acid-polypeptide condensates, sulfuric acid esters, alkyl sulfates, ethoxylated alkyl sulfates, ester linked sulfonates, alpha olefin sulfonates, phosphated ethoxylated alcohols, and mixtures thereof. In an embodiment, the surfactant is sodium lauryl sulfate (SLS) (also known as sodium laurylsulfate or sodium dodecyl sulfate (SDS or NaDS)) with a molecular formula: C₁₂H₂₅SO₄Na.

Examples of cationic surfactants include, but are not limited to, monoalkyl quaternary ammonium salts, dialkyl quaternary ammonium compounds, amidoamines, aminimides, and mixtures thereof.

Examples of amphoteric surfactants include, but are not limited to, N-substituted alkyl amides, N-alkyl betaines, sulfobetaines, N-alkyl-β-aminoproprionates, and mixtures thereof.

Examples of non-ionic surfactants include, but are not limited to, ethoxylated alcohols, ethoxylated esters, ethoxylated amides, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glyceryl esters, sorbitan esters, sucrose esters, glucose (dextrose) esters, and mixtures thereof.

In an embodiment, the polymers utilized in the present invention are hydrophilic polymers. Examples of hydrophilic polymers include, but are not limited to, polyethylene glycols (PEG), polyvinylpyrrolidones (PVP), polyvinylalcohols (PVA), vinylpyrrolidone-vinyl acetate copolymers, hypromellose (HPMC), Poloxamers, carboxymethylcellulose (CMC), hydroxypropylmethylcellulose acetate succinate (HPMCAS), hydroxypropylcellulose (HPC), polyacrylates, polymethacrylates, urea and sugar. Hydrophobic polymers such as polyesters and polyimides may also be used, as well as blends of hydrophilic and hydrophobic polymers. In an embodiment, the polymer used is a hypromellose.

The solvents utilized in the present invention can be selected from the group consisting of an aqueous solvent, alcohol, ketone, ester, ether, aliphatic hydrocarbon, halogenated solvent, cyclaliphatic, aromatic, heterocyclic and a mixture thereof. Examples of solvents include, but are not limited to, water, acetone, diacetone, alcohol, methanol, ethanol, isopropanol (IPA), n-propanol, n-butanol, methyl acetate, ethyl acetate, isopropyl acetate, butyl acetate, methyl isobutyl ketone, methyl propyl ketone, hexane, heptane ethylene glycol monoethyl ether, ethylene glycol monoethyl acetate, methylene dichloride, ethylene dichloride, propylene dichloride, carbon tetrachloride, chloroform, nitroethane, nitropropane, tetrachoroethan, ethyl ether, diethyl ether, isopropyl ether, 1,4-dioxane, cyclohexane, cyclooctane, benzene, toluene, naphtha, tetrahydrofuran (THF), dichloromethane (DCM), acetonitrile (MeCN), dimethylformamide (DMF), dimethyl sulfoxide (DMSO), acetic acid, formic acid, diglyme and a mixture thereof. In an embodiment, the solvent used to form solution 1 is acetone/purified water at a ratio of about 4:1.

Any suitable seal coating material may be used for coating the active seeds, including, but not limited to, Eudragit® polymers (including, for example, a Eudragit® E acrylic polymer), hydroxypropylmethylcellulose (HPMC), hydroxypropyl cellulose (HPC), polyethylene glycol, polyvinyl alcohol (PVA) and the like.

Additionally, an enteric coating may be applied to the formulation if desired. Any suitable enteric coating material may be used, including, but not limited to, cellulose acetate phthalate; hydroxypropyl methylcellulose phthalate (HPMCP); hydroxypropyl cellulose acetyl succinate; polyvinyl acetate phthalate; copolymerized methacrylic acid/methacrylic acid methyl esters, such as Eudragit® L 12.5, Eudragit® L 100-55, and Eudragit® S 100; and mixtures thereof.

Pharmaceutical dosage forms of the present invention include, but are not limited to, those suitable for oral, rectal, topical and buccal (e.g. sublingual) administration. Formulations suitable for oral administration may be presented in discrete units, such as capsules, cachets, lozenges or tablets, each containing a predetermined amount of a disclosed solid dispersion; or as a powder or granules. As used herein, “tablet” refers to a pharmaceutical dosage form comprising a mixture of active substances and excipients, usually in powder form, pressed or compacted into a solid. The excipients can include binders, glidants (flow aids) and lubricants to ensure efficient tabletting; disintegrants to promote tablet break-up in the digestive tract; sweeteners or flavours to enhance taste; and pigments to make the tablets visually attractive. A polymer coating is often applied to make the tablet smoother and easier to swallow, to control the release rate of the active ingredient, to make it more resistant to the environment (extending its shelf life), or to enhance the tablet's appearance. In the tablet pressing process, all the ingredients should be well-mixed. If a sufficiently homogenous mix of the components cannot be obtained with simple blending processes, the ingredients can be granulated prior to compression to assure an even distribution of the active compound in the final tablet. Two basic techniques are used to granulate powders for compression into a tablet: wet granulation and dry granulation. Powders that can be mixed well do not require granulation and can be compressed into tablets through direct compression. A tablet may be prepared by compressing or molding the solid dispersion of the present invention with one or more excipients. Compressed tablets may be prepared by compressing, in a suitable machine, the solid dispersion in a free-flowing form optionally mixed with a binder, lubricant, inert diluent and/or surface active/dispersing ingredient(s). A capsule can be prepared by filling a suitable gelatine capsule with a therapeutically effective amount of the fenofibrate solid dispersion

Pharmaceutical dosage forms suitable for buccal (sub-lingual) administration include lozenges comprising the solid dispersion in a flavored base, usually sucrose and acacia or tragacanth, and pastilles comprising the solid dispersion in an inert base such as gelatin and glycerin or sucrose and acacia.

Pharmaceutical dosage forms suitable for rectal administration can be prepared by mixing the solid dispersion with one or more conventional solid carriers, for example, cocoa butter, and then shaping the resulting mixture.

Pharmaceutical dosage forms suitable for topical application to the skin can be in the form of an ointment, cream, lotion, paste, gel, spray, aerosol, or oil. Carriers which may be used include Vaseline, lanolin, polyethylene glycols, alcohols, and combinations thereof.

The active ingredients of the present invention may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms, are described in the Handbook of Pharmaceutical Excipients, American Pharmaceutical Association (1986), incorporated herein by reference in its entirety. Examples of pharmaceutically acceptable carriers include water, ethanol, polyols, vegetable oils, fats, waxes, polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulfate, talc, as well as high molecular weight polyethylene glycols. The artisan of ordinary skill in the art will recognize that many different excipients can be used in formulations according to the present invention and the list provided herein is not exhaustive.

Suitable diluents that are useful in the present invention include, e.g., lactose USP; lactose USP, anhydrous; lactose USP, spray dried; starch USP; directly compressible starch; mannitol USP; sorbitol; dextrose monohydrate; microcrystalline cellulose NF; dibasic calcium phosphate dihydrate NF; sucrose-based diluents; confectioner's sugar; monobasic calcium sulfate monohydrate; calcium sulfate dihydrate NF; calcium lactate trihydrate granular NF; dextrates NF (e.g., Emdex™); dextrose (e.g., Cerelose™); inositol; hydrolyzed cereal solids such as the Maltrons™ and Mor-Rex™; amylose; powdered cellulose (e.g., Elcema™); calcium carbonate; glycine; bentonite; polyvinylpyrrolidone; and the like.

Suitable disintegrants can be selected from starches; sodium starch glycolate; clays (such as Veegum™ HV); celluloses (such as purified cellulose, methylcellulose, sodium carboxymethycellulose, and carboxymethylcellulose); alginates; pre-gelatinized corn starches (such as National™ 1551 and National™ 1550); crospovidone USP NF; and gums (such as agar, guar, locust bean, pectin, and tragacanth).

Suitable binding ingredients and adhesives can be selected from acacia; tragacanth; sucrose; gelatin; glucose; starch; cellulose materials such as, but not limited to, methylcellulose and sodium carboxymethylcellulose (e.g., Tylose™); alginic acid and salts of alginic acid; magnesium aluminum silicate; polyethylene glycol: guar gum; polysaccharide acids; bentonites; polyvinylpyrrolidone (povidone); polymethacrylates; hydroxypropyl methylcellulose (HPMC or hypromellose); hydroxypropyl cellulose (Klucel™); ethyl cellulose (Ethocel™); pregelatinized starch (such as National™ 1511 and Starch 1500). In an embodiment, the binder is hypromellose. Hypromellose is commercially available in various grades with differing viscosities and chemistry. In an embodiment, low-viscosity Hypromellose type 2910, with a viscosity of 3 cPs, is used in order to minimize the viscosity of the coating solution (for ease of processing) while maintaining an adequate binding ability.

Suitable lubricants can be selected from glyceryl behenate (Compritol™ 888); metallic stearates (e.g., magnesium, calcium and sodium stearates); stearic acid; hydrogenated vegetable oils (e.g., Sterotex™); talc; waxes; boric acid; sodium benzoate and sodium acetate; sodium chloride; DL-Leucine; polyethylene glycols (e.g., Carbowax™ 4000 and Carbowax™ 6000); sodium oleate; sodium benzoate; sodium acetate; sodium lauryl sulfate; sodium stearyl fumarate (Pruv™); and magnesium lauryl sulfate.

Suitable anti-adherents can be selected from talc, cornstarch, colloidal silicone dioxide (Cab-O-Sil™), DL-Leucine, sodium lauryl sulfate, and metallic stearates.

Additionally, colorants and opacifiers may also be included in the active ingredient solution to be spray coated in order to improve the appearance and other characteristics. Colorants and opacifiers which may be used include, e.g., water soluble dyes, water insoluble pigments and natural colorants, such as D&C and FD&C Blue, Red and Yellow lakes and dyes. The amount of colorant used depends upon the appearance desired and can be adjusted accordingly. Pigments including titanium dioxide, calcium carbonate, calcium sulfate, magnesium oxide, magnesium carbonate, aluminum silicate, aluminum hydroxide, talc and iron oxide may also be used. Metal oxides may be used as opacifiers, such as titanium dioxide.

The poor water solubility of fenofibrate can limit its absorption in the gastrointestinal (GI) tract. To remedy this problem, research groups have tried a multitude of strategies including, for example, micronized fenofibrate formulations, the combination of fenofibrate and vitamin E, the use of diethylene glycol monoethyl ether (DGME) as solubilizer, and the combination of fenofibrate with one or more polyglycolyzed glycerides. Another approach includes employing nanoparticulate fenofibrate. The pharmacokinetics parameters for nanoparticulate fenofibrate formulations, commercially available from Abbott as TriCor® 145 mg and 48 mg, are reportedly not significantly affected by the fed or fasting state of the subject. Another approach involves using “Controlled agglomeration” by incorporating the fenofibrate into a “meltable” vehicle, commercially available from Sciele Pharma, Inc. as Fenoglide® 120 mg and 40 mg.

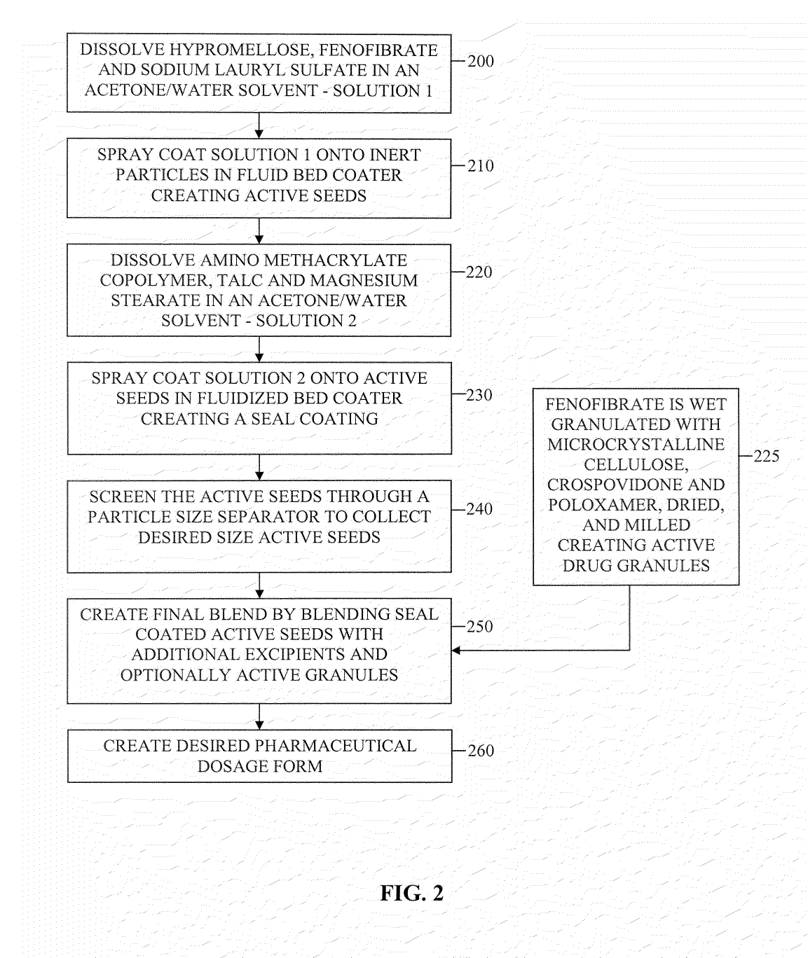

One aspect of the present invention is directed to fenofibrate pharmaceutical dosage forms that are bioequivalent to currently marketed fenofibrate pharmaceutical dosage forms. In an embodiment, a method of preparing a fenofibrate solid dispersion of the present invention and a pharmaceutical dosage form comprising the solid dispersion is illustrated in FIG. 2. As illustrated in FIG. 2, hypromellose, fenofibrate, and sodium lauryl sulfate are dissolved in an acetone/purified water solvent to form solution 1, step 200. In an embodiment, the hypromellose is low viscous hypromellose/HPMC—Pharmacoat® 603 with a viscosity of 3 centipoise (cps).

In an embodiment, the fenofibrate is micronized fenofibrate. The dissolution profile of the fenofibrate in solution 1 can be adjusted by varying the amount of hypromellose and sodium lauryl sulfate included. For example, there is a direct relationship between the amount of hypromellose and dissolution of the fenofibrate. Therefore, as the ratio of hypromellose to fenofibrate increases, the apparent solubility and bioavailability also increases. In certain embodiments, the ratio of hypromellose to fenofibrate is from about 20:1 to about 1:20; from about 15:1 to about 1:15; from about 10:1 to about 1:10; from about 5:1 to about 1:5; from about 2:1 to about 1:2; from about 1.5:1 to about 1:1.5. In an embodiment, the ratio of polymer to API is 1.5:1.

Using a fluid bed bottom spray coating process (Wurster coating), solution 1 is coated onto sugar spheres and dried, creating active seeds, step 210. Amino methacrylate copolymer (Eudragit® E 100), talc and magnesium stearate are dissolved in an acetone/purified water solvent to form solution 2, step 220. The active seeds are then coated and dried with solution 2 to create a seal coating, step 230. The active seeds are then screened through a particle size separator to collect the desired size active seeds, step 240. In an embodiment, active seeds between 40 mesh (about 420 μm, 0.42 mm) and 60 mesh (about 250 μm, 0.25 mm) are collected. In an embodiment, the size of the active seeds are about 0.30 mm. The method may optionally include step 225, where micronized fenofibrate having a particle size specification of 90% less than 15 microns undergoes a wet granulation process (WG) with microcrystalline cellulose, crospovidone and Poloxamer, to create active granules. In step 250, a final blend is created by blending the active seeds with pharmaceutical excipients and optionally the active granules. A desired pharmaceutical dosage form can be created, step 260. In an embodiment, step 225 is performed to adjust the bioavailability of the final blend, and thus the pharmaceutical dosage form. In an embodiment, the addition of step 225 to the method results in the reduction in the bioavailability of the final blend, and thus the pharmaceutical dosage form.

In an embodiment, the pharmaceutical dosage form comprises a plurality of drug-coated seeds and a plurality of drug granules in a single matrix tablet. In an embodiment, the pharmaceutical dosage form is an immediate release tablet. In an embodiment, the drug granules are present in the pharmaceutical dosage form at a percentage ranging from about 20% to about 80%; from about 30% to about 70%; from about 35% to about 65%; from about 40% to about 60%. In an embodiment, the drug granules are present in the pharmaceutical dosage form at a percentage of about 60%. By varying the percentage of drug granules in the pharmaceutical dosage pharmacokinetic parameters can be altered. In an embodiment, the overall absorption rate, Cₘₐₓ, under fasting state decreases as the percentage of micronized drug increases.

In an embodiment, a pharmaceutical dosage form includes a solid dispersion prepared by dissolving fenofibrate, hypromellose (as binder) and sodium lauryl sulfate (as solubilizer) in an acetone/water solution; coating the solution onto inert particles via a fluidized-bed coater to create active seeds; granulating fenofibrate, microcrystalline cellulose and crospovidone (as compression aid), and Poloxamer (as solubilizer) in a high shear granulator with purified water followed by drying and milling to create active granules; blending the active seeds, the active granules and optionally additional excipients together to form a final blend; and creating the pharmaceutical dosage form. The preparation of the solid dispersion may further include sealing the active seeds with a seal coat. The sealing may be accomplished by dissolving Eudragit E100, talc (as anti-adherent) and magnesium stearate in an acetone/water solution and coating the solution onto the active seeds.

In some embodiments, a solid dispersion of the present invention can include more than one API. The additional API can be administered in the same dosage form (during a method of manufacturing as described herein). The second API can be in the same dosage form or administered in a different dosage form The second API can be, for example, adjunctive therapy for treating hypercholesterolemia or an ingredient to treat hypertension.

Examples of cholesterol lowering ingredients that can be combined with fenofibrate in the present invention include, but are not limited to, resins (bill acid binders or bile acid sequestrants), such as cholestyramine, colestipol, colesevelam; other fibrates, such as ciprofibrate, gemfibrozil and bezafibrate; statins, such as atorvastatin, fluvastatin, lovastatin, itavastatin, mevastatin, pitavastatin, pravastatin, rosuvastatin, and simvastatin; cholesterol absorption inhibitors, such as ezetimibe (Zetia®); squalene synthetase inhibitors; cholesterol ester transfer protein inhibitors, such as torcetrapib; niacin and omega-3 fatty acid.

Examples of antihypertensives that can be combined with fenofibrate in the present invention include, but are not limited to, beta-blockers such as alprenolol, atenolol, timolol, pindolol, propranolol, metoprolol, bisoprololfumerate, esmolol, acebutelol, myeloprolol, acebutolol, betaxolol, celiprolol, nehivolol, tertatolol, oxprenolol, amusolalul, carvedilol and labelalol; ACE (angiotensin converting enzyme) inhibitors such as quinapril, lisinopril, enalapril, captopril, benazepril, perindopril, trandolapril, Ibsinopril, ramipril, cilazapril, delapril, imidapril, moexipril, spirapril, temocapril, zofenopril, fasidotril omapatrilat and gemopatrilat; calcium channel blockers such as nifedipine, felodipine, nicardipine, isradipine, nimodipine, diltiazem, amlodipine, nitrendipine, verapamil, lacidipine, lercanidipine, aranidipine, cilnidipine, clevidipine, azelnidipine, barnidipine, efonodipine, iasidipine, lemildipine, iereanidipinc, manidipine, nilvadipinc, pranidipinc and furnidipine; alpha-blockers such as doxazosin, urapidil, prazosin, terazosin, and bunazosin; diuretics such as thiazides/sulphonamides (e.g. bendroflumetazide, chlorothalidone, hydrochlorothiazide and clopamide), loop-diuretics (e.g. bumetanide, furosemide and torasemide) and potassium sparing diuretics (e.g. amiloride, spironolactone); renin inhibitors such as aliskiren and vasopressin; angiotensin antagonists such as irbesartan, candesartancilexetil, losartan, valsartan, telmisartan, eprosartan, candesartan, eprosartan, iosartan, olmesartan and pratosartan, indapainicles, guanylate cyclase stimulators, hydralazines, methyldopa, docarpamine and moxonidine.

The present invention is described in the following Examples, which are set forth to aid in the understanding of the invention, and should not be construed to limit in any way the scope of the invention as defined in the claims which follow thereafter. The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1

### Polymer Screening with Polarized Microscope

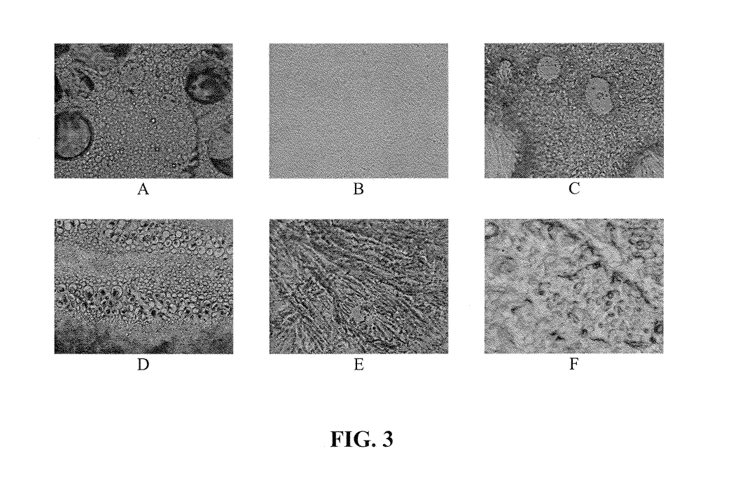

0.58 grams of polymer A (low viscous hypromellose/HPMC—Pharmacoat® 603), polymer B (vinylpyrrolidone-vinyl acetate copolymer (Copovidon)—Kollidon® VA 64 PVP), polymer C (PEG 6000), polymer D (hydroxypropylcellulose—Klucel® HPC Grade LF), polymer E (Poloxamer 188—Poloxamer with a polyoxypropylene molecular mass of 1,800 g/mol and a 80% polyoxyethylene content), and polymer F (hydroxypropylmethylcellulose acetate succinate —HPMCAS) were each dissolved in 14.4 grams of water/acetone (1:4) in 20 mL glass vials. Then 0.15 grams of sodium lauryl sulfate and 0.58 grams of fenofibrate were dissolved in each vial. Two drops of each solution were added to a microscope slide. The slides were dried in a 30° C. oven and then observed with a Nikon Eclipse 50i polarized microscope. FIGS. 3A-3F show polarized micrographs depicting the morphology of the drug/polymer films. Films with Klucel® HPC Grade LF (FIG. 3D) and HPMCAS (FIG. 3F) showed large drug crystals which are typically not suitable for preparing a solid dispersion of the present invention.

### Example 2

### Polymer Screening with Dissolution Testing

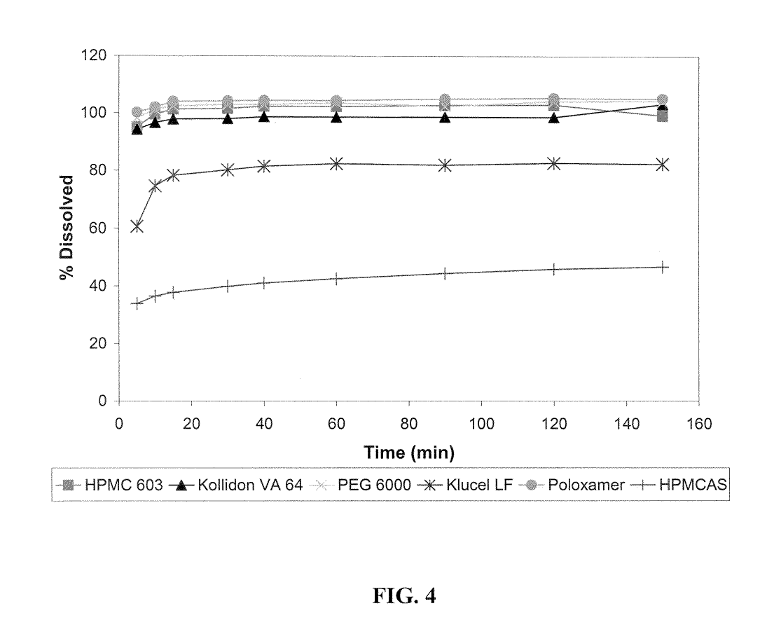

Dissolution testing was conducted using the USP II paddle method at 50 rpm, 1000 mL of Simulated Gastric Fluid (SGF) in 1% Tween 80 pH 1.2 at about 37° C. For all tests, 4 grams of the solution prepared in Example 1 was added to the SGF. The concentration of the fenofibrate was measured using High Performance Liquid Chromatography (HPLC) with an ultraviolet (UV) detector at 285 nm. FIG. 4 shows the dissolution profiles of the various dosage forms. The results indicate that Pharmacoat® 603, Kollidon® VA 64 PVP, PEG6000 and Poloxamer 188 significantly improved the dissolution of fenofibrate compared with Klucel® HPC Grade LF and HPMCAS.

### Example 3

### Active-Coating on Sugar Spheres

348 grams of Pharmacoat® 603, 58.4 grams of sodium lauryl sulfate, and 232 grams of fenofibrate were dissolved in a mixture of 1150 grams of water and 4600 grams of acetone to create solution 1. Solution 1 was coated on sugar spheres with 60-80 mesh size in a Glatt-Powder-Coater-Granulator (GPCG-1). The solution spray rate was in the range of about 2 g/min to about 15 g/min and the product temperature was controlled at about 30° C. After all coating solution was consumed, the coated seeds were dried at inlet air temperature at about 45° C. for about 15 minutes. Then, the coated seeds were passed through a 40-mesh screen and then through a 60 mesh screen. The seeds between 40 mesh and 60 mesh were collected.

### Example 4

### Seal-Coating of Active-Coated Seeds

24 grams of Basic Butylated Methacrylate Copolymer (Eudragit® E100) was dissolved in a mixture of 16 grams of water, 109 grams of acetone, and 164 grams of isopropyl alcohol. Then 8.0 grams of Talc was dispersed in the solution. The mixture was coated onto 800 grams of the active-coated seeds from Example 3 in a GPCG-1. The spray rate was in the range of about 6 g/min to about 10 g/min and the product temperature was controlled at about 26° C. After all coating suspension was consumed, the seal-coated seeds were dried at inlet air temperature of about 55° C. for about 20 minutes. Then, the seal-coated seeds were passed through a 40-mesh screen and then through a 60 mesh screen. The seeds between 40 mesh and 60 mesh were collected.

### Example 5

### Final Blending and Compression of Tablet A

171.8 grams of Avicel® HFE-102, 53.1 grams of Avicel® PC 105, 28.5 grains of Pregelatinized Starch, 2.8 grains of Syloid® 244 FP, 57.1 grams of Poloxamer 338, and 28.5 grams of Crospovidone were mixed together and passed through a 30 mesh screen. The mixture was blended with 228.8 grams of seal-coated seeds from Example 4 for about 5 minutes and further blended with 1.4 grams of stearic acid for about 1 minute. The final blend was compressed into tablets with target tablet weight of 1625.0 mg and target hardness of 18 kp using a Jenn Chiang Machinery (JCMCO) Rotary Tablet Press.

### Example 6

### Final Blending and Compression of Tablet B

171.8 gams of Avicel® HFE-102, 81.7 grams of Avicel® PC 105, 28.5 grams of Pregelatinized Starch, 2.8 grams of Syloid® 244 FP, 28.5 grams of Poloxamer 407, and 28.5 grains of Crospovidone were mixed together and passed through a 30-mesh screen. The mixture was blended with 228.8 grams of seal-coated seeds from Example 4 for about 5 minutes and further blended with 1.4 grams of stearic acid for about 1 minute. The final blend was compressed into tablets with target tablet weight of 1625.0 mg and target hardness of 18 kp using a Jenn Chiang Machinery (JCMCO) Rotary Tablet Press.

### Example 7

### In-Vitro Dissolution Testing of Seeds and Tablets

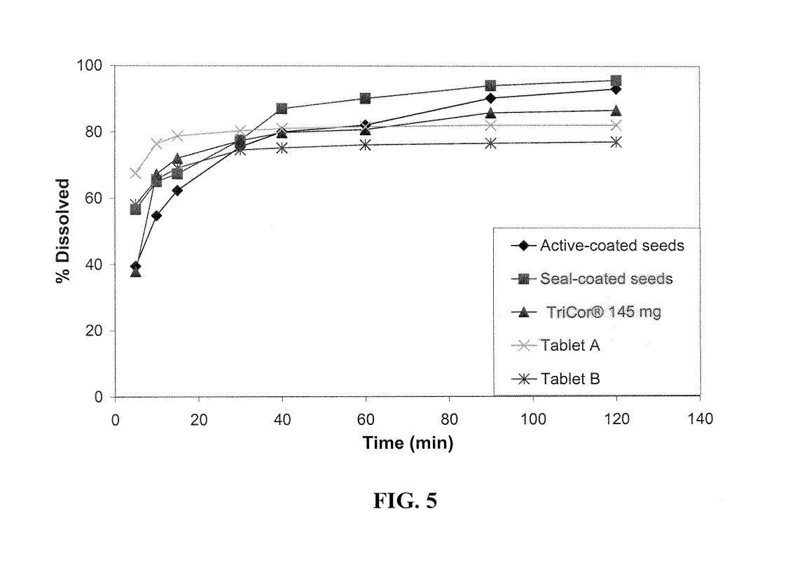

Dissolution testing of seeds (Example 3 and 4) and tablets (Example 5 and 6) was conducted using the USP II paddle method at 50 rpm, 1000 mL of Simulated Gastric Fluid (SGF) in 1% Tween 80 pH 1.2 at about 37° C. Dosage forms equivalent to 145 mg fenofibrate (625 mg active-coated seeds, 650 mg seal-coated seeds, Tablet A, Tablet B, or Tricor® 145 mg) were added to the SGF. The concentration of the fenofibrate was measured using HPLC with a UV detector at 285 nm. FIG. 5 shows the dissolution profiles of the various dosage forms. The seal-coated seeds, Tablet A, and Tablet B exhibited higher initial dissolution rate than Tricor® 145 mg tablet, indicating faster dissolution than the nanocrystal formulation. Seal-coated seeds have faster dissolution than active-coated seeds, suggesting seal-coating may be required to protect active-coated formulations.

### Example 8

### In-Vivo Plasma Concentration Under Fasted State

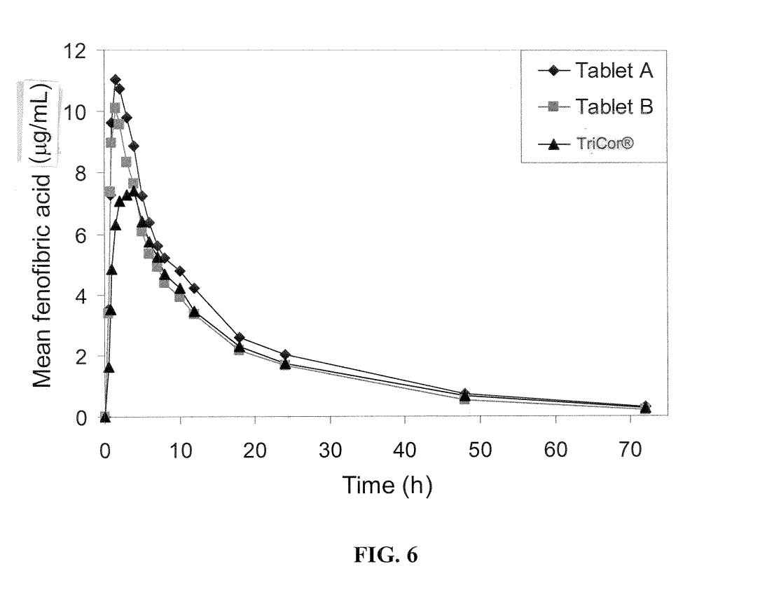

Tablet A, B and Tricor® 145 mg were dosed to 12 healthy volunteers with three-way crossover design under fasting conditions, see FIG. 6. Compared with Tricor® 145 mg tablets, Tablet A and Tablet B showed significantly higher Cₘₐₓ and comparable AUC data. The Tablet A/Tricor® ratio of Ln(Cₘₐₓ) was 1.20 and Tablet B/Tricor® ratio of Ln(Cₘₐₓ) was 1.27 respectively. The Tablet A/Tricor® ratio of Ln(AUC, observed) was 1.06 and Tablet B/Tricor® ratio of Ln(AUC, observed) was 1.11 respectively. The Tₘₐₓ for Tablet A and Tablet B was 1.5 hours and the Tₘₐₓ for Tricor® was 4 hours, indicating Tablet A and Tablet B showed significantly faster absorption than Tricor®.

### Example 9

### In-Vivo Plasma Concentration Under Fed State

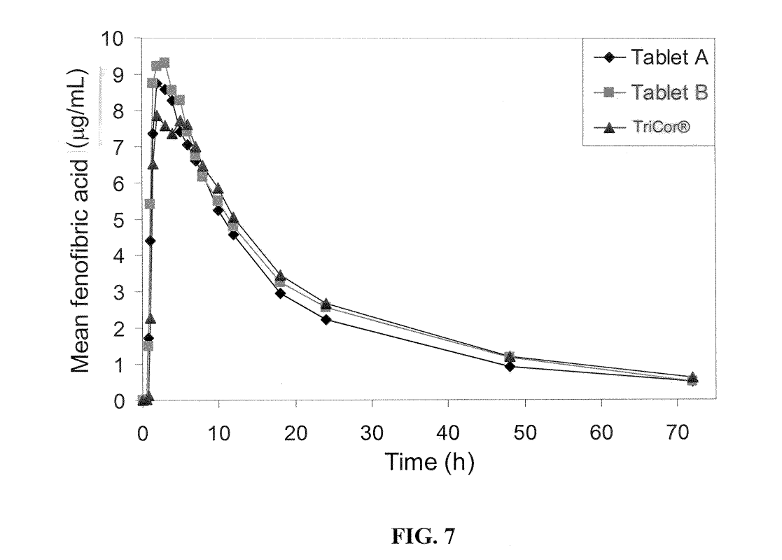

Tablet A, B and Tricor® 145 mg were dosed to 12 healthy volunteers with three-way crossover design under fed conditions, see FIG. 7. Compared with Tricor® 145 mg tablets, Tablet A and Tablet B showed comparable Cₘₐₓ and AUC. The Tablet A/Tricor® ratio of Ln(Cₘₐₓ) is 0.88 and Tablet B/Tricor® ratio of Ln(Cₘₐₓ) is 1.03 respectively. The Tablet A/Tricor ratio of Ln(AUC, observed) is 0.95 and Tablet B/Tricor® ratio of Ln(AUC, observed) is 0.98 respectively. As evidenced by Example 8 and 9, the pharmacokinetic profile of Tablet A and Table B are not significantly affected by the fed or fasted state of a subject ingesting the composition.

### Example 10

### Pharmaceutical Dosage Form Comprising Nanosized Fenofibrate

1. 7.0 grams of Sodium lauryl sulfate and 3.3 grams of Hypromellose Pharmacoat® 603 were dissolved into 40 grams of Purified water to create a solution.
2. Charge 496.4 grams of fenofibrate nanomaterial (provided by NanoMaterial Technology, Singapore) and 22.0 grams of Crospovidone into a high shear granulator.
3. Granulate the material in step 2 with the solution of step 1. After the solution was consumed, granulate the wet mixture with 43 grams of purified water.
4. Discharge the granules from step 3 and dry at 40° C. for about 20 hours.
5. Pass the dried granules in step 4 through a Fitzmill equipped with 20 mesh.
6. Blend 488.2 grams of the milled granules in step 5 with 112.1 grams of Crospovidone, 58.7 grams of Avicel® PH-102, 1.2 grams of Colloidal silicon dioxide, and 3.1 grams of sodium stearyl fumarate for 10 minutes in a blender.
7. The final blend in step 6 was compressed into a tablets containing 120 mg fenofibrate with a (JCMCO) Rotary Tablet Press.

### Example 11

### Pharmaceutical Dosage Form Comprising Drug-Coated Particles

1. 101.14 kg of Pharmacoat® 603, 16.97 kg of sodium lauryl sulfate, and 67.43 kg of fenofibrate were dissolved in a mixture of 334.2 kg of water and 1336.95 kg of acetone. The solution was coated on 105.09 kg sugar spheres with 60-80 mesh size in a Glatt-Powder-Coater-Granulator (GPCG-60). The solution spray rate was in the range of about 200 g/min to about 1200 g/min and the product temperature was controlled at about 30° C. After all coating solution was consumed, the coated seeds were dried at inlet air temperature at about 45° C. for about 15 minutes.
2. 8.37 kg of Basic Butylated Methacrylate Copolymer (Eudragit® E100) was dissolved in a mixture of 8.37 kg of water, 142.3 kg of acetone. Then 6.51 kg of Talc and 1.86 kg of Magnesium Stearate were dispersed in the solution. The mixture was coated onto the active-coated seeds in step 2 in a GPCG-60. The spray rate was in the range of about 200 g/min to about 1200 g/min and the product temperature was controlled at about 26° C. After all coating suspension was consumed, the seal-coated seeds were dried at inlet air temperature of about 45° C. for about 15 minutes. Then, the seal-coated seeds were passed through a 40-mesh screen and then through a 60 mesh screen. The seeds between 40 mesh and 60 mesh were collected.
3. Charge 7.5 kg of Microcrystalline Cellulose (Avicel PH-301), 1.5 kg of Crospovidone, and 4 kg of Poloxamer 407 into a high shear granulator. Granulate the material with 3.5 kg of Purified Water. Discharge the granules and dry at 40° C. for about 20 hours. Pass the dried granules in step 4 through a Fitzmill equipped with 20 mesh.
4. 1458.8 grams of active seeds from step 2, 229.6 grams of granules from step 3, 471.0 grams of Microcrystalline Cellulose (Avicel® PH-200), 186 grams of Microcrystalline Cellulose (Avicel® PH-105), 74.0 grams of Crospovidone, 6.0 grams of Silicon Dioxide (Syloid® 244 FP) were blended in a blender for 30 minutes. Then the blend was blended with 6.0 grams of Magnesium Stearate for 5 minutes to obtain the final blend.
5. The final blend in step 4 was compressed into a tablets containing 120 mg fenofibrate with a (JCMCO) Rotary Tablet Press.

### Example 12

### Pharmaceutical Dosage Form Comprising Fenofibrate-Coated Particles and 25% Fenofibrate Granules

1. Charge 4.188 kg of Fenofibrate Micronized USP, 2.736 kg of Microcrystalline Cellulose (Avicel PH-301), 1.380 kg of Crospovidone, and 3.696 kg of Poloxamer 407 into a high shear granulator. Granulate the material with 1.52 kg of water. Discharge the granules and dry at 40° C. for about 20 hours. Pass the dried granules through a Fitzmill equipped with 20 mesh.
2. 411.0 grams of active seeds from step example 10 step 2, 86.0 grams of granules from step 1, 893.8 grams of Microcrystalline Cellulose (Avicel® PH-200), 496 grams of Microcrystalline Cellulose (Avicel® PH-105), 198.6 grams of Crospovidone, 16.6 grams of Silicon Dioxide (Syloid® 244 FP) were blended in a blender for 30 minutes. Then the blend was blended with 16.6 grams of Magnesium Stearate for 5 minutes to obtain the final blend.
3. The final blend in step 2 was compressed into a tablets containing 120 mg fenofibrate with a (JCMCO) Rotary Tablet Press.

### Example 13

### Pharmaceutical Dosage Form Comprising Fenofibrate-Coated Particles and 40% Fenofibrate Granules

1. Charge 167.4 grams of Fenofibrate Micronized USP, 5.7 grams of Microcrystalline Cellulose (Avicel PH-301), 34.5 grams of Crospovidone and 92.4 grams of Poloxamer 407 into a high shear granulator. Granulate the material with 70.6 grams of water. Discharge the granules and dry at 40° C. for about 20 hours. Pass the dried granules through a Fitzmill equipped with 20 mesh.
2. 328.8 grams of active seeds from step example 10 step 2, 86.0 grams of granules from step 1, 553.2 grams of Microcrystalline Cellulose (Avicel® PH-200), 186.0 grams of Microcrystalline Cellulose (Avicel® PH-105), 74.0 grams of Crospovidone, 6.0 grams of Silicon Dioxide (Syloid® 244 FP) were blended in a blender for 30 minutes. Then the blend was blended with 6.0 grams of Magnesium Stearate for 5 minutes to obtain the final blend.
3. The final blend in step 2 was compressed into a tablets containing 120 mg fenofibrate with a (JCMCO) Rotary Tablet Press.

### Example 14

### Pharmaceutical Dosage Form Comprising Fenofibrate-Coated Particles and 65% Fenofibrate Granules

1. 7.22 kg of Pharmacoat® 603, 1.22 kg of sodium lauryl sulfate, and 4.80 kg of fenofibrate were dissolved in a mixture of 23.82 kg of water and 95.27 kg of acetone. The solution was coated on 30.02 kg sugar spheres with 60-80 mesh size in a Glatt-Powder-Coater-Granulator (GPCG-30). The solution spray rate was in the range of about 50 g/min to about 350 g/min and the product temperature was controlled at about 30° C. After all coating solution was consumed, the coated seeds were dried at inlet air temperature at about 45° C. for about 15 minutes.
2. 10.336 kg of Pharmacoat® 603, 1.7354 kg of sodium lauryl sulfate, and 6.8803 kg of fenofibrate were dissolved in a mixture of 34.11 kg of water and 136.45 kg of acetone. The solution was coated on 20.66 kg of active-coated seeds in step 1 in a Glatt-Powder-Coater-Granulator (GPCG-30). The solution spray rate was in the range of about 50 g/min to about 350 g/min and the product temperature was controlled at about 30° C. After all coating solution was consumed, the coated seeds were dried at inlet air temperature at about 45° C. for about 15 minutes.
3. 1.1391 kg of Basic Butylated Methacrylate Copolymer (Eudragit® E100) was dissolved in a mixture of 1.1372 kg of water and 19.36 kg of acetone. Then 0.8868 kg of Talc and 0.2523 kg of Magnesium Stearate were dispersed in the solution. The mixture was coated onto the active-coated seeds in step 2 in a GPCG-30. The spray rate was in the range of about 50 g/min to about 350 g/min and the product temperature was controlled at about 26° C. After all coating suspension was consumed, the seal-coated seeds were dried at inlet air temperature of about 45° C. for about 15 minutes. Then, the seal-coated seeds were passed through a 40-mesh screen and then through a 60 mesh screen. The seeds between 40 mesh and 60 mesh were collected.
4. Charge 0.5824 kg of Fenofibrate Micronized USP, 2.7776 kg of Microcrystalline Cellulose (Avicel PH-301), 1.3664 kg of Crospovidone and 1.9787 grams of Poloxamer 407 into a high shear granulator. Granulate the material with 1.65 kg of water. Repeat the granulation process 2 times to make total three sublots. Discharge the granules and dry at 40° C. for about 20 hours. Pass the dried granules through a Fitzmill equipped with 20 mesh.
5. 23.02 kg of active seeds from step 3, 19.20 kg of granules from step 4, 73.94 kg of Microcrystalline Cellulose (Avicel® PH-200), 22.32 kg of Microcrystalline Cellulose (Avicel® PH-105), 8.88 kg of Crospovidone, 0.72 kg of Silicon Dioxide (Syloid® 244 FP) were blended in a blender for 30 minutes. Then the blend was blended with 0.72 kg of Magnesium Stearate for 5 minutes to obtain the final blend.
6. The final blend in step 5 was compressed into a tablets containing 120 mg fenofibrate with a (JCMCO) Rotary Tablet Press.

### Example 15

### Pharmaceutical Dosage Form Comprising Fenofibrate-Coated Particles and 60% Fenofibrate Granules

Active-Coating: A bottom spray coating (Wurster coating) process was chosen for the active coating process that yields the active seeds. The Wurster process results in highly uniform coating of particulates. Active seeds were completed with two stage coating (11.1% and 21.9%)

### Active Seeds Coating (I), 11.1%

1) Active-solution preparation: Dissolve Fenofibrate, Hypromellose and Sodium Lauryl Sulfate into a mixture of Purified Water and Acetone.
2) Coat the solution from Step 1 onto Sugar Spheres in the Fluidized-Bed Coater (with Wurster insert).

### Active Seeds Coating (II), 21.9%

3) Active-solution preparation: Dissolve Fenofibrate, Hypromellose and Sodium Lauryl Sulfate into a mixture of Purified Water and Acetone.
4) Seal-coat suspension preparation: Dissolve Eudragit E100 into mixture of Purified Water and Acetone. Disperse Talc and Magnesium Stearate into the solution.
5) Coat the solution from Step 3 onto Active Seeds (I) from step 2 in the Fluidized-Bed Coater (with Wurster insert).
6) Coat the suspension from Step 4 onto seeds from Step 5 in the Fluidized-Bed Coater (with Wurster insert).
7) Screen Active Seeds through a Particle Size Separator using 35-mesh and 60-mesh screens. Collect seeds retained on the 60-mesh screen for further processing.

| Process | | |
|---|---|---|
| Parameters | Experimental Batch | Pivotal batch |
| Equipment: | 5.5″ Wurster/1 gun | 18″ Wurster/1 gun |
| Partitions/Number | | |
| of Spray Guns | | |
| Batch Load | 300-650 g | 44 kg |
| Fluidizing air | N/A | 190-300 |
| volume (cfm) | | |
| Inlet air | 35-44 (active coating) | 35-46 (active coating) |
| temperature (° C.) | 30-36 (seal coating) | 24-35 (seal coating) |
| Product/Outlet | 27-31 (active coating) | 29-31 (active coating) |
| temperature (° C.) | 26-29 (seal coating) | 26-29 (seal coating) |
| Atomizing air | 1.5-2.5 | 1.5-2.5 |
| pressure (bar) | | |
| Nozzle size (mm) | 0.8 | 1.5 |
| Spray rate (g/min) | 2-14 | 50-330 |

Active Granulation/Milling: Fenofibrate, Microcrystalline Cellulose, Crospovidone and Poloxamer 407 were granulated with Purified Water in a high shear mixer. After drying, the granules were milled through Fitzmill with 20-mesh screen.
8) Charge Microcrystalline Cellulose (PH 301), Fenofibrate, Crospovidone and Poloxamer into a high shear granulator and dry-mix the ingredients.
9) Granulate using Purified Water.
10) Use additional Purified Water if required until the granules have the consistency of wet snow.
11) Dry the granules in an oven until LOD is NMT 3.0%.
12) Mill the dried granules through a Fitzmill equipped with 20 mesh screen.
   Blending: The Active-Coated Seeds and Active Granules were blended with Microcrystalline Cellulose, Crospovidone, Silicon Dioxide and Magnesium Stearate in a blender until uniformity was achieved.
13) Screen Microcrystalline Cellulose (PH 105) and Silicon Dioxide through a Particle-size separator equipped with a 30 mesh screen.
14) Charge the screened material from Step 13, Active Seeds from Step 7, Active Granules from Step 12, Microcrystalline Cellulose (PH 200) and Crospovidone into a slant cone blender. Blend for 30 minutes.
15) Screen the Magnesium Stearate through a 30 mesh screen and add to the blender. Continue blending until uniformity is achieved and then discharge the blend.
   Compression: This blend was then compressed into tablets on a rotary tablet press.
16) Compress the final blend at pre-determined parameters using a Rotary Tablet Press.

| | % (w/w) | | mg/unit | |
|---|---|---|---|---|
| Ingredients | 40 mg | 120 mg | 40 mg | 120 mg |

Active-Coated Seeds

| | | | | |
|---|---|---|---|---|
| Fenofibrate, Micronized, USP | 3.87 | 3.87 | 16.00 | 48.00 |
| Hypromellose, type 2910, USP | 5.82 | 5.82 | 24.04 | 72.12 |
| Sodium Lauryl Sulfate, NF | 0.98 | 0.98 | 4.04 | 12.12 |
| Sugar Spheres, NF, Mesh: 60-80 | 6.05 | 6.05 | 25.01 | 75.04 |
| Amino Methacrylate Copolymer, | 0.48 | 0.48 | 1.99 | 5.96 |
| NF (Eudragit E 100) | | | | |
| Talc, USP | 0.37 | 0.37 | 1.55 | 4.64 |
| Magnesium Stearate, NF | 0.11 | 0.11 | 0.44 | 1.32 |
| Purified Water, USP | — | — | — | — |
| Acetone, NF | — | — | — | — |

Active Granules

| | | | | |
|---|---|---|---|---|
| Fenofibrate, Micronized, USP | 5.81 | 5.81 | 24.00 | 72.00 |
| Microcrystalline Cellulose, | 3.48 | 3.48 | 14.40 | 43.20 |
| NF (PH-301) | | | | |
| Crospovidone, NF | 1.48 | 1.48 | 6.10 | 18.30 |
| Poloxamer 407, NF | 2.14 | 2.14 | 8.83 | 26.50 |
| Purified Water, USP | — | — | — | — |

Final Blend

| | | | | |
|---|---|---|---|---|
| Microcrystalline Cellulose, | 47.48 | 47.48 | 196.27 | 588.80 |
| NF (PH-200) | | | | |
| Microcrystalline Cellulose, | 15.00 | 15.00 | 62.00 | 186.00 |
| NF (PH-105) | | | | |
| Crospovidone, NF | 5.97 | 5.97 | 24.67 | 74.00 |
| Silicon Dioxide, NF | 0.48 | 0.48 | 2.00 | 6.00 |
| Magnesium Stearate, NF | 0.48 | 0.48 | 2.00 | 6.00 |
| Total | 100.0 | 100.0 | 413.3 | 1240.0 |

### Example 16

### In-Vitro Dissolution Testing

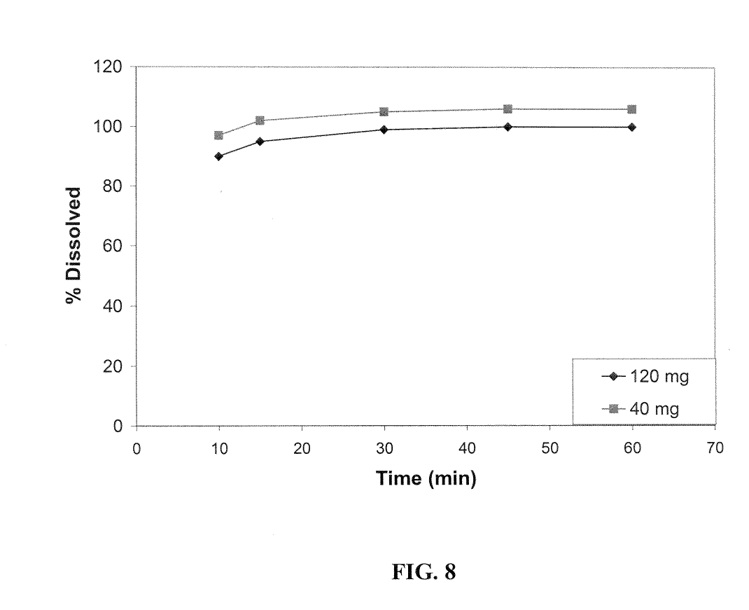

Dissolution testing of the 40 mg and the 120 mg tablets from Example 15 was conducted using the USP paddle method at 75 rpm, 900 mL of medium with 0.75% of sodium lauryl sulfate in water at about 37° C. The concentration of the fenofibrate was measured using HPLC with a UV detector at 285 nm. FIG. 8 shows the dissolution profiles of the various dosage forms. Greater than 85% release of the drug was observed at 10 minutes for both strengths.

### Example 17

### In-Vivo Plasma Concentration

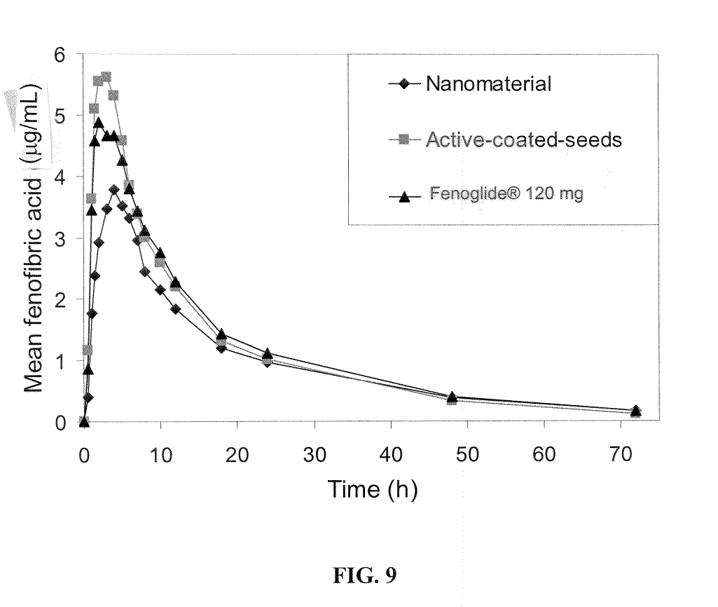

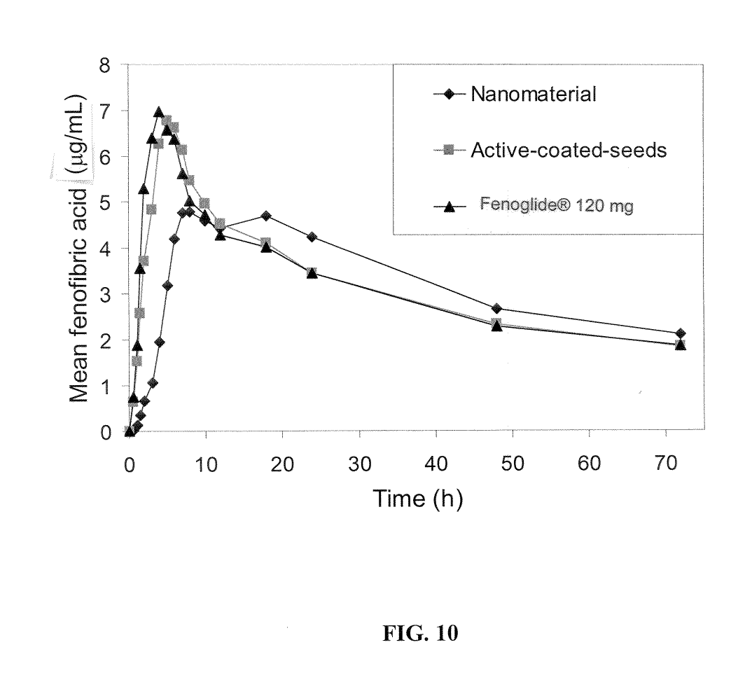

Nanomaterial of fenofibrate prepared as described in Example 10, active-coated-seeds prepared as described in Example 11 and Fenoglide® 120 mg were dosed to 12 healthy volunteers with three-way crossover design under fasting and fed conditions, see FIG. 9 and FIG. 10. The nanosized fenofibrate was granulated with 1.0% Sodium Lauryl Sulfate (SLS). The active-coated-seeds were blended with 2.1% Poloxamer 407 as granules. The final blends of the two platforms were compressed into a tablet dosage form. The results are summarized in the table below:

| | | |
|---|---|---|
| Pilot study | Nanomaterial | Active-coated-seeds |

FED (n = 12)

| | | |
|---|---|---|
| Ln (AUCₜ) ratio | 105.5 | 104.7 |
| Ln (Cmax) ratio | 78.0 | 102.7 |

FASTED (n = 12)

| | | |
|---|---|---|
| Ln (AUCₜ) ratio | 87.3 | 109.2 |
| Ln (Cmax) ratio | 77.5 | 141.1 |

The formulation with nanomaterial showed lower Cₘₐₓ than Fenoglide® under both fasted and fed states. The formulation with active-coated-seeds showed comparable AUC and Cₘₐₓ to Fenoglide® under fed state, however, fasting Cₘₐₓ was 41% higher than Fenoglide®.

A second set of studies designed also as a three-way crossover with two new test products, active-coated-seeds were combined with micronized fenofibrate to reduce the absorption rate under fasting condition. The micronized fenofibrate was granulated with Poloxamer and blended with active-coated-seeds. Two levels of percentage of micronized fenofibrate (25% and 40%) as described in Examples 12 and 13, were evaluated. The results are summarized in the table below:

| | | |
|---|---|---|
| Pilot study | 40% micronized API | 25% micronized API |

FED (n = 12)

| | | |
|---|---|---|
| Ln (AUCₜ) ratio | 97.3 | 96.8 |
| Ln (Cmax) ratio | 90.5 | 97.4 |

FASTED (n = 12)

| | | |
|---|---|---|
| Ln (AUCₜ) ratio | 105.9 | 113.5 |
| Ln (Cmax) ratio | 120.8 | 126.8 |

Both formulations are bioequivalent to Fenoglide® under fed state. The overall absorption rate, Cₘₐₓ, under fasting state decreases with percentage of micronized fenofibrate as expected. The fasting Cₘₐₓ with 40% micronized fenofibrate was still 20% higher than Fenoglide®.

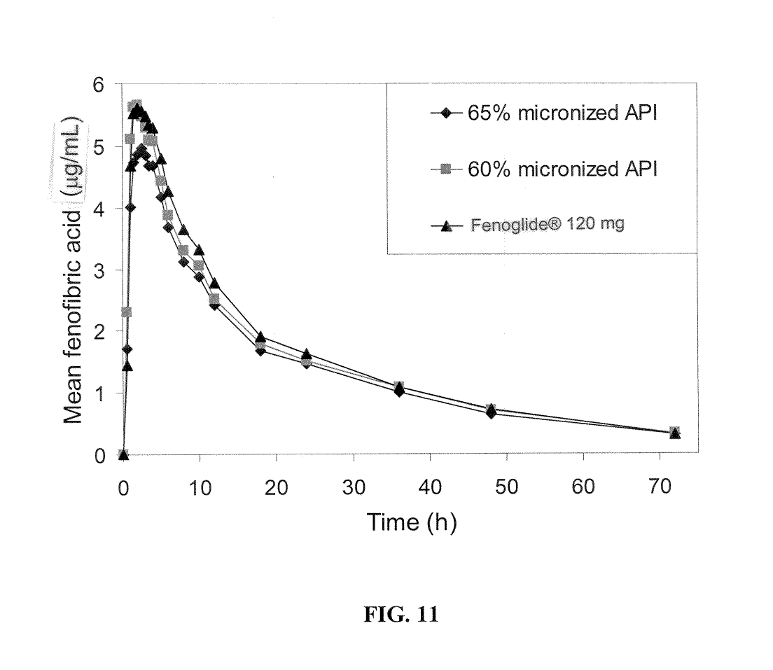

A third set of studies designed also as a three-way crossover with two new test products under fasting state with two levels of percentage of micronized fenofibrate (60% and 65%) as described in Examples 14 and 15, were evaluated. Both formulations exhibit the similar fasting Cₘₐₓ and AUC as Fenoglide®. The results are summarized in FIG. 11 and the table below:

| | | |
|---|---|---|
| Pivotal study | 65% micronized API | 60% micronized API |

FASTED (n = 23)

| | | |
|---|---|---|
| Ln (AUCₜ) ratio | 86.5 | 93.0 |
| Ln (Cmax) ratio | 88.2 | 99.0 |

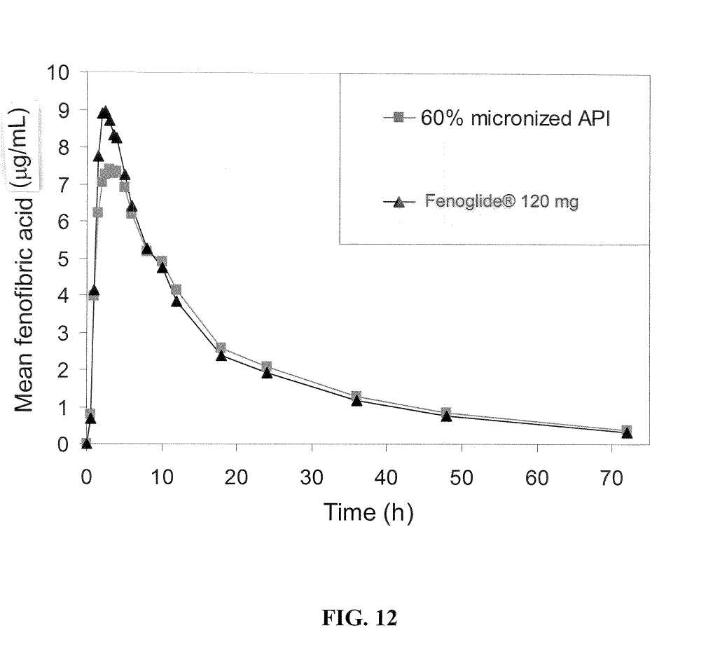

A fourth set of studies designed as a two-way crossover with the 60% micronized API formulation under fed state was conducted. The results are summarized in FIG. 12 and the table below:

| | |
|---|---|
| Pivotal study | 60% micronized API |

FED (n = 22)

| | |
|---|---|
| Ln (AUCₜ) ratio | 97.8 |
| Ln (Cmax) ratio | 80.2 |

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

What is claimed is:

1. A solid dispersion comprising:
a plurality of coated particles comprising inert particles with a coating,
wherein the coating comprises fenofibrate dispersed in a hydrophilic polymer, and
wherein the inert particles comprise nonpareils; and
a plurality of granules comprising micronized fenofibrate with at least one pharmaceutically acceptable excipient.

2. The solid dispersion of claim 1 wherein the hydrophilic polymer of the coating is selected from the group consisting of polyethylene glycol, polyvinylpyrrolidone, polyvinylalcohol, vinylpyrrolidone-vinyl acetate copolymers, hypromellose, hydroxypropylmethylcellulose acetate succinate, hydroxypropylcellulose, Poloxamer, polyacrylate, polymethacrylate, urea and sugar.

3. The solid dispersion of claim 2 wherein the hydrophilic polymer of the coating is hypromellose.

4. The solid dispersion of claim 3 wherein a ratio of the hypromellose to fenofibrate in the coating is from about 10:1 to about 1:10.

5. The solid dispersion of claim 1 wherein the granules are present at a percentage ranging from about 0% to about 60%, by weight, based on the weight of the fenofibrate.

6. The solid dispersion of claim 1 wherein the fenofibrate of the coating is further dispersed in a surfactant.

7. The solid dispersion of claim 6 wherein the surfactant is sodium lauryl sulfate.

8. The solid dispersion of claim 1 wherein the coated particles are further coated with a seal coating.

9. The solid dispersion of claim 1 wherein the plurality of granules are present in the solid dispersion at a percentage ranging from about 5% to about 65%.

10. A pharmaceutical dosage form comprising the solid dispersion of claim 1.

11. The pharmaceutical dosage form of claim 10 in a unit dose comprising from about 10 mg to about 200 mg fenofibrate.

12. The pharmaceutical dosage form of claim 10 in a unit dose comprising from about 10 mg to about 200 mg fenofibrate in the form of a single matrix tablet.

13. The pharmaceutical dosage form of claim 10 in a unit dose comprising from about 10 mg to about 200 mg fenofibrate in the form of an immediate release tablet.

14. The pharmaceutical dosage form of claim 13 wherein at least 85% of the total weight of fenofibrate in the solid dispersion is dissolved within 10 minutes when tested with a USP paddle method at 75 rpm, 900 mL of medium with 0.75% of sodium lauryl sulfate in water at about 37° C.

15. The pharmaceutical dosage form of claim 13 which provides a mean Cₘₐₓ of fenofibric acid of about 4.0 μg/mL to about 8.0 μg/mL after administration of a single dose to a patient population in the fasted state based on a 120 mg dose of fenofibrate.

16. The pharmaceutical dosage form of claim 13 which provides a mean Cₘₐₓ of fenofibric acid of about 5.0 μg/mL to about 9.0 μg/mL after administration of a single dose to a patient population in the fed state based on a 120 mg dose of fenofibrate.

17. The pharmaceutical dosage form of claim 10 comprising a sufficient amount of granules in order to decrease relative bioavailability of fenofibrate as compared to a pharmaceutical dosage form that does not include granules.

18. A method of treating hypercholesterolemia in a patient comprising administering the pharmaceutical dosage form of claim 13.

19. A method of treating hypertriglyceridemia in a patient comprising administering the pharmaceutical dosage form of claim 13.

20. A method of preparing a solid dispersion comprising:
dissolving an insoluble active pharmaceutical ingredient and a hydrophilic polymer in a suitable solvent to form a solution;
preparing a plurality of coated particles by spray coating a plurality of inert particles with the solution using a fluid bed coating process;
preparing a plurality of granules by wet granulating a mixture of an insoluble active pharmaceutical ingredient with at least one pharmaceutical acceptable excipient; and
blending the plurality of coated particles and the plurality of granules.

21. The method of claim 20 wherein the insoluble active pharmaceutical ingredient in the solution and the insoluble pharmaceutical ingredient in the mixture are of a same type.

22. The method of claim 20 wherein the insoluble active pharmaceutical ingredient in the solution and the insoluble pharmaceutical ingredient in the mixture are in a micronized form.

23. The method of claim 20 wherein the insoluble active pharmaceutical ingredients are selected from the group consisting of analgesics, anti-inflammatory ingredients, anthelmintics, anti-arrhythmic ingredients, antibiotics, anticoagulants, antihypercholesterolemia ingredients, antidepressants, antidiabetic ingredients, antiepileptics, antihistamines, antihypertensive ingredients, antimuscarinic ingredients, antimycobacterial ingredients, zmtineoplastic ingredients, immunosuppressants, antithyroid ingredients, antiviral ingredients, anxiolytic sedatives, astringents, beta-adrenoceptor blocking ingredients, blood products and substitutes, cardiac inotropic ingredients, contrast media, corticosteroids, cough suppressants, diagnostic ingredients, diagnostic imaging ingredients, diuretics, dopaminergics, haemostatics, itninuriological ingredients, lipid regulating ingredients, muscle relaxants, parasympathomimetics, parathyroid calcitonin and biphosphonates, prostaglandins, radio-pharmaceuticals, sex hormones, anti-allergic ingredients, stimulants and anorctics, sympathotnimetics, thyroid ingredients, vasodilators, and xanthines.

24. The method of claim 23 wherein the insoluble active pharmaceutical ingredients are antihypercholesterolemia ingredients.

25. The method of claim 20 wherein the insoluble active pharmaceutical ingredients are fibrates selected from the group consisting of bezafibrate, ciprofibrate, clofibrate, gemfibmzil, fenofibrate and pharmaceutically acceptable salts thereof.

26. The method of claim 25 wherein the insoluble active pharmaceutical ingredients are fenofibrate.

27. The method of claim 20 wherein the hydrophilic polymer in the solution is selected from the group consisting of polyethylene glycol, polyvinylpyrrolidone, polyvinylalcohol, vinylpyrrolidone-vinyl acetate copolymers, hypromellose, hydroxypropylmethylcellulose acetate succinate, hydroxypropylcellulose, Poloxamer, polyacrylate, polymethacrylate, urea and sugar.

28. The method of claim 20 wherein the solution further includes a surfactant.

29. The method of claim 28 wherein the surfactant is selected from the group consisting of an anionic surfactant, a cationic surfactant, an amphoteric surfactant, a non-ionic surfactant and a mixture thereof.

30. The method of claim 28 wherein the surfactant is sodium lauryl sulfate.

31. The method of claim 20 wherein the coated particles are further coated with a seal coating.

32. The method of claim 20 wherein the plurality of granules are present in the solid dispersion at a percentage ranging from about 35% to about 65%.

33. A solid dispersion comprising:
a plurality of coated particles comprising inert particles with a coating,
wherein the coating comprises at least one insoluble active pharmaceutical ingredient dispersed in a hydrophilic polymer, and
wherein the inert particles comprise nonpareils; and
a plurality of granules comprising at least one insoluble active pharmaceutical ingredient with at least one pharmaceutically acceptable excipient.

34. The solid dispersion of claim 33 wherein the hydrophilic polymer of the coating is selected from the group consisting of polyethylene glycol, polyvinylpyrrolidone, polyvinylalcohol, vinylpyrrolidone-vinyl acetate copolymers, hypromellose, hydroxypropylmethylcellulose acetate succinate, hydroxypropylcellulose, Poloxamer, polyacrylate, polymethacrylate, urea and sugar.

35. The solid dispersion of claim 34 wherein the hydrophilic polymer of the coating is hypromellose.

36. The solid dispersion of claim 35 wherein a ratio of the hypromellose to insoluble active pharmaceutical ingredient in the coating is from about 10:1 to about 1:10.

37. The solid dispersion of claim 33 wherein the granules are present at a percentage ranging from about 0% to about 60%, by weight, based on the weight of the active pharmaceutical ingredient.

38. The solid dispersion of claim 33 wherein the insoluble active pharmaceutical ingredient of the coating is further dispersed in a surfactant.

39. The solid dispersion of claim 38 wherein the surfactant is sodium lauryl sulfate.

40. The solid dispersion of claim 33 wherein the coated particles are further coated with a seal coating.

41. The solid dispersion of claim 33 wherein the plurality of granules are present in the solid dispersion at a percentage ranging from about 5% to about 65%.

42. The solid dispersion of claim 33 wherein the insoluble active pharmaceutical ingredient of the coating and the insoluble pharmaceutical ingredient of the granules are of a same type.

43. The solid dispersion of claim 33 wherein the insoluble active pharmaceutical ingredient of the coating and the insoluble pharmaceutical ingredient of the granules are in a micronized form.

44. The solid dispersion of claim 33 wherein the insoluble active pharmaceutical ingredients are selected from the group consisting of analgesics, anti-inflammatory ingredients, anthelmintics, anti-arrhythmic ingredients, antibiotics, anticoagulants, antihypercholesterolemia ingredients, antidepressants, antidiabetic ingredients, antiepileptics, antihistamines, antihypertensive ingredients, antimuscarinic ingredients, antimycobacterial ingredients, zmtineoplastic ingredients, immunosuppressants, antithyroid ingredients, antiviral ingredients, anxiolytic sedatives, astringents, beta-adrenoceptor blocking ingredients, blood products and substitutes, cardiac inotropic ingredients, contrast media, corticosteroids, cough suppressants, diagnostic ingredients, diagnostic imaging ingredients, diuretics, dopaminergics, haemostatics, itninuriological ingredients, lipid regulating ingredients, muscle relaxants, parasympathomimetics, parathyroid calcitonin and biphosphonates, prostaglandins, radio-pharmaceuticals, sex hormones, anti-allergic ingredients, stimulants and anorctics, sympathotnimetics, thyroid ingredients, vasodilators, and xanthines.

45. The solid dispersion of claim 33 wherein the insoluble active pharmaceutical ingredients are antihypercholesterolemia ingredients.

46. The solid dispersion of claim 33 wherein the insoluble active pharmaceutical ingredients are fibrates selected from the group consisting of bezafibrate, ciprofibrate, clofibrate, gemfibmzil, fenofibrate and pharmaceutically acceptable salts thereof.

47. A pharmaceutical dosage form comprising the solid dispersion of claim 33.

48. The pharmaceutical dosage form of claim 47 in a unit dose comprising from about 10 mg to about 200 mg insoluble active pharmaceutical ingredient.

49. The pharmaceutical dosage form of claim 47 in a unit dose comprising from about 10 mg to about 200 mg insoluble active pharmaceutical ingredient in the form of a single matrix tablet.

50. The pharmaceutical dosage form of claim 47 in a unit dose comprising from about 10 mg to about 200 mg insoluble active pharmaceutical ingredient in the form of an immediate release tablet.

51. The pharmaceutical dosage form of claim 50 wherein at least 85% of the total weight of insoluble active pharmaceutical ingredient in the solid dispersion is dissolved within 10 minutes when tested with a USP paddle method at 75 rpm, 900 mL of medium with 0.75% of sodium lauryl sulfate in water at about 37° C.

52. The pharmaceutical dosage form of claim 47 comprising a sufficient amount of granules in order to decrease relative bioavailability of the insoluble active pharmaceutical ingredient as compared to a pharmaceutical dosage form that does not include granules.
